SEQUENCE LISTING (3 sequences)

SEQ ID NO: 1 - DNA, 1418 bp, Homo Sapien
gggcgggcccaccgcgacaccgcgccgccaccccgaccccgctgcgcacggcctgtccgctgcacaccagcttgttggcgtcttcgtcgccgcgctcgccccgggctactcctgcgcgccacaatgagctcccgcatcgccagggcgctcgccttagtcgtcacccttctccacttgaccaggctggcgctctccacctgccccgctgcctgccactgccccctggaggcgcccaagtgcgcgccgggagtcgggctggtccgggacggctgcggctgctgtaaggtctgcgccaagcagctcaacgaggactgcagcaaaacgcagccctgcgaccacaccaaggggctggaatgcaacttcggcgccagctccaccgctctgaaggggatctgcagagctcagtcagagggcagaccctgtgaatataactccagaatctaccaaaacggggaaagtttccagcccaactgtaaacatcagtgcacatgtattgatggcgccgtgggctgcattcctctgtgtccccaagaactatctctccccaacttgggctgtcccaaccctcggctggtcaaagttaccgggcagtgctgcgaggagtgggtctgtgacgaggatagtatcaaggaccccatggaggaccaggacggcctccttggcaaggagctgggattcgatgcctccgaggtggagttgacgagaaacaatgaattgattgcagttggaaaaggcagctcactgaagcggctccctgtttttggaatggagcctcgcatcctatacaaccctttacaaggccagaaatgtattgttcaaacaacttcatggtcccagtgctcaaagacctgtggaactggtatctccacacgagttaccaatgacaaccctgagtgccgccttgtgaaagaaacccggatttgtgaggtgcggccttgtggacagccagtgtacagcagcctgaaaaagggcaagaaatgcagcaagaccaagaaatcccccgaaccagtcaggtttacttacgctggatgtttgagtgtgaagaaataccggcccaagtactgcggttcctgcgtggacggccgatgctgcacgccccagctgaccaggactgtgaagatgcggttccgctgcgaagatggggagacattttccaagaacgtcatgatgatccagtcctgcaaatgcaactacaactgcccgcatgccaatgaagcagcgtttcccttctacaggctgttcaatgacattcacaaatttagggactaaatgctacctgggtttccagggcacacctagacaaacaagggagaagagtgtcagaatcagaatcatggagaaaatgggcgggggtggtgtgggtgatgggactcattgtagaaaggaagccttctcattcttgaggagcattaaggtat

SEQ ID NO: 2 - protein, 381 aa, Homo Sapien [sequence omitted]

SEQ ID NO: 3 - protein, 11 aa, Homo Sapien
RLFNDIHKFRD

(19) United States
(12) Patent Application Publication
(10) Pub. No.: US 2004/0023910 A1
(43) Pub. Date: Feb. 5, 2004

(54) Use of cyr61 in the treatment and diagnosis of human uterine leiomyomas

(76) Inventors: Zhiming Zhang, Malvern, PA (US); Deepak Sampath, Freehold, NJ (US); Yuan Zhu, Norristown, PA (US); Richard Winneker, Penllyn, PA (US)

Correspondence Address: WYETH, PATENT LAW GROUP, FIVE GIRALDA FARMS, MADISON, NJ, 07940, US

(21) Appl. No.: 10/381,644

(22) PCT Filed: Sep. 28, 2001
(86) PCT No.: PCT/US01/30783

(51) Int. Cl. A61K 48/00

(52) U.S. Cl. 514/44; 424/93.2

(57) ABSTRACT

The present invention relates to methods of inhibiting uterine leiomyoma proliferation and preventing formation of uterine leiomyomas, compounds and compositions that stimulate induction of the Cyr61 gene and compounds that increase Cyr61 activity. The present invention also relates to methods of screening ligands that regulate Cyr61 protein expression. The invention further relates to methods of diagnosing patients with uterine leiomyomas associated with a downregulation of Cyr61 expression. The invention also describes antibodies and related pharmaceutical compositions.

### PRIORITY

This application claims priority under 35 U.S.C. § 119 from provisional patent application Serial No. 60/236,887, filed Sep. 29, 2001; which is hereby incorporated by reference in its entirety.

### FIELD OF THE INVENTION

The present invention relates to methods of inhibiting uterine leiomyoma proliferation and preventing formation of uterine leiomyomas, compounds and compositions that stimulate induction of the Cyr61 gene and compounds that increase Cyr61 activity. The present invention also relates to methods of screening ligands that regulate Cyr61 protein expression. The invention further relates to methods of diagnosing patients with uterine leiomyomas associated with a downregulation of Cyr61 expression. The invention also describes antibodies and pharmaceutical compositions related thereto. Transgenic animals are also contemplated by the present invention.

### BACKGROUND OF THE INVENTION

Uterine leiomyomas, or fibroids, are the most common tumors of the reproductive tract afflicting women between the ages of 30-55 years. Little is known of the etiology and mechanisms of pathogenesis in leiomyomas. Uterine leiomyomas are typically defined as benign tumors of the myometrial smooth muscle tissue. Leiomyoma cells are believed to originate from dedifferentiated smooth muscle cells in the myometrium that exhibit elevated mitotic activity as a result of clonal expansion (Rein and Nowak, Sem. Reprod. Endocrinol., 1992,10:310-319). Although considered to be a benign disease, uterine leiomyomas account for greater than 30% of all hysterectomies performed in the United States and pose a major health concern among women (Cramer, Sem. Reprod. Endocrinol., 1992,10:320-324.).

An emerging group of growth factor-regulated immediate-early genes that play a role in development, cell proliferation, and tumorogenesis belongs to the CCN (CTGF/Cyr61/Cef10/NOVH) family. All CCN proteins (1) display a high degree of conservation among family members and across species; (2) are cysteine-rich and structurally similar to extracellular matrix-associated molecules; (3) are composed of multifunctional modular domains; and (4) have been shown to mediate a variety of cell functions such as cell adhesion, cell migration, mitogenesis, cell survival, and differentiation (Law and Lam, Experimental Cell Res, 1999, 248:44).

Cyr61 is a secreted, cysteine-rich heparin-binding CCN protein that associates with the cell surface and the extracellular matrix. Specifically, Cyr61 has been shown to be involved in developmentally regulated processes including angiogenesis and chondrogenesis. The Cyr61 protein possesses many biochemical features that resemble the Wnt-1 protein and other growth factors (Yang and Law, Cell Growth & Diff, 1991, 2:351). The human Cyr61 protein is 381 amino acids in length with a molecular mass of about 42 kilo-daltons (kDa). See FIG. 1 and PCT Application No. WO 97/339950. The human Cyr61 gene is localized in the short arm of chromosome 1 (1p22-31) (Charles et al., Oncogene, 1991, 8:23; Jay et al., Oncogene, 1997, 14:1753), and the gene was identified by differential hybridization screening of a cDNA library of serum-stimulated BALB/c3T3 fibroblasts (See FIG. 2 and Law and Nathans, P.N.A.S., 1987, 84:1182). Comparison of the human and murine Cyr61 sequences indicates that they are 91% similar (PCT Publication No. WO 97/339950). It was previously shown that Cyr61 protein expression is upregulated in stage II invasive ductal carcinoma breast cancer (U.S. Provisional Patent Application No. 60/213,182, filed Jun. 21, 2000).

Integrins are heterodimeric transmembrane receptors that are non-covalently associated in the presence of divalent cations. Several integrins, but not all, interact with adhesive ligands through recognition of a canonical Arg-Gly-Asp (RGD) binding motif present in a subset of extracellular matrix proteins and can initiate signaling pathways commonly shared by growth factors or cytokines. Cyr61, which lacks the RGD motif, has been shown to bind directly to two integrins: αᵥβ₃ and α_{IIn}β₃ (Kireeva et al., J. Biol. Chem., 1998, 273:3090; Babic et al., Mol. Cell. Biol., 1999,19:2958; Jedsadayanmata et al., J. Biol. Chem., 1999,274:24321). Integrin αᵥβ₃ has been shown to be directly involved in angiogenesis in vivo and regulate tumor metastasis. Therefore, the effects of Cyr61 as an angiogenic factor is proposed to be mediated in part by αᵥβ₃ . Recent studies have shown that human platelets bind to Cyr61 in an activation dependent manner via α_{IIb}β₃ (Jedsadayarmnata et al., 1999). Murine Cyr61 has been shown to also bind α₆β₁, an integrin primarily expressed in fibroblasts, and heparin sulfate proteoglycans in a co-receptor fashion (Chen et al., J. Biol. Chem., 2000, 275:24953). Binding to both receptors is critical for fibroblast adhesion in vitro. Mutagenesis studies has identified the C-terminal domain (a.a. 250-354) as absolutely required for binding to α₆β₁. Thus, it is evident that Cyr61 is an integrin ligand based on its location within the extracellular matrix and its ability to directly associate with them.

The present inventors have found that detection and regulation of Cyr61 expression and activities is useful in the prevention, diagnosis, and treatment of uterine leiomyomas.

### SUMMARY OF THE INVENTION

The present invention provides methods for inhibiting uterine leiomyoma proliferation. Methods can comprise increasing the level of mRNA encoding Cyr61, increasing translation of Cyr61 mRNA, upregulating expression of Cyr61 protein, increasing the activity of Cyr61 protein, or increasing the level of Cyr61 protein in leiomyoma tissues. The present invention also provides methods for preventing uterine leiomyoma in normal myometrial tissue. Methods can comprise maintaining a uterine leiomyoma preventing level of mRNA encoding Cyr61, translation activity of Cyr61, expression of Cyr61 protein, activity of Cyr61 protein, or affinity of Cyr61 for basic fibroblast growth factor or heparin binding epidermal growth factor.

These methods include, but are not limited to, delivery of the Cyr61 protein to the cell, administration of an expression vector encoding the Cyr61 protein, and administration of a therapeutically effective amount of a compound that modulates binding of Cyr61 to intracellular proteins (e.g., integrin receptors).

Also provided are antibodies that recognize a portion or all of the Cyr61 protein. These antibodies may be polyclonal or monoclonal, chimeric or hurnanized, and/or anti-idiotypic. Preferably, these antibodies do not recognize or bind proteins that belong to the same protein family as Cyr61.

The present invention further provides methods for diagnosing uterine leiomyomas. Methods include those which comprise comparing the level of Cyr61 in a cell in suspect tissue to the level of Cyr61 in normal myometrium tissue that is autologous to the suspect tissue. A lower level of Cyr61 in the suspect tissue than in the normal tissue indicates the presence of uterine leiomyoma in the suspect tissue. The level of Cyr61 in this method can be determined by exposing the suspect and normal tissue to a Cyr61 antibody which recognizes the Cyr61 protein, and then comparing the amount of antibody bound by each tissue. A lower level of bound antibody in the suspect tissue than in the normal tissue indicates the presence of uterine leiomyoma in the suspect tissue.

Methods for screening compounds that inhibit or prevent proliferation of uterine leiomyoma also are provided. These methods comprise comparing the amount of Cyr61 expressed by leiomyoma cells exposed to a compound, to the amount of Cyr61 expressed by uterine leiomyomas not exposed to the compound. A greater level of Cyr61 expressed in cells exposed to the compound compared to cells not exposed to the compound indicates that the compound may inhibit or prevent uterine leiomyomas.

Transgenic non-human animals that have a uterus and that comprise DNA such as, for example, human DNA, which can be induced to overexpress Cyr61 in the uterus also are contemplated by the present invention.

Methods for detecting the presence of uterine leiomyomas are also contemplated in the present invention. These methods comprise comparing the level of Cyr61 mRNA or protein from suspect uterine leiomyoma tissue to the level of Cyr61 mRNA or protein from normal myometrium tissue. A lower level of Cyr61 mRNA or protein in the suspect uterine leiomyoma tissue compared to the normal myometrium tissue indicates the presence of uterine leiomyoma.

A kit for diagnosing uterine leiomyoma is contemplated by the present invention. The lit includes an antibody that recognizes or binds to Cyr 61.

The present invention further contemplates expression vectors comprising the nucleic acid depicted in FIG. 6 operably associated with an expression control sequence. In specific embodiments, the expression control sequence may be an estrogen response element or abasic fibroblast growth factor response element. The heparin binding epidermal growth factor also regulates Cyr61 expression. Pharmaceutical compositions comprising the vector or the protein and methods for preventing and inhibiting proliferation of uterine leiomyomas using the pharmaceutical compositions also are contemplated.

The present invention provides methods for inhibiting uterine leiomyomas proliferation. Methods can comprise administering to a subject in need of treatment an amount of a compound effective to stimulate the synthesis of mRNA encoding Cyr61, the translation of Cyr61 mRNA, the expression of Cyr61, or the activity of Cyr61 protein. The present invention also contemplates increasing the total level of Cyr61 protein in leiomyoma tissues. In certain embodiments, compounds that inhibit uterine leiomyomas also downregulate the synthesis of IGF I and/or IGF II mRNA, translation of IGF I and/or IGF II mRNA, expression of IGF I and/or IGF II protein, the activity of IGF I and/or IGF II, the synthesis of basic fibroblast growth factor and/or heparin binding epidermal growth factor mRNA, translation of basic fibroblast growth factor and/or heparin binding epidermal growth factor mRNA, expression of basic fibroblast growth factor and/or heparin binding epidermal growth factor protein, or the activity of basic fibroblast growth factor and/or heparin binding epidermal growth factor.

The invention further provides methods for preventing uterine leiomyoma in myometrial tissue. Methods include those which comprise administering to a subject in need of preventing uterine leiomyoma in myometrial tissue an amount of a compound effective to maintain a uterine leiomyoma preventing level of of mRNA encoding Cyr61, translation activity of Cyr61 mRNA, expression of Cyr61, or activity of Cyr61 protein in myometrial tissues. In certain embodiments, the compound decreases estrogen receptor activity.

The present invention further provides methods of preventing proliferation of uterine leiomyomas, where the method comprises administering to a subject an amount of a compound effective to increase the affinity of Cyr61 protein for basic fibroblast growth factor or heparin binding epidermal growth factor. In certain embodiments, the compound decreases estrogen receptor activity.

Methods for preventing uterine leiomyomas also include those which comprise administering to a subject in need of preventing uterine leiomyomas, an amount of a compound effective to also maintain a uterine leiomyoma preventing level of synthesis of IGF I and/or IGF II mRNA, translation of IGF I and/or IGF II mRNA, expression of IGF I and/or IGF II protein, or the activity of IGF I and/or IGF II. Methods for preventing uterine leiomyomas include those which comprise administering to a subject in need of preventing uterine leiomyomas, an amount of a compound also effective to maintain a uterine leiomyoma preventing level of the synthesis of basic fibroblast growth factor and/or heparin binding epidermal growth factor mRNA, translation of basic fibroblast growth factor and/or heparin binding epidermal growth factor mRNA, expression of basic fibroblast growth factor and/or heparin binding epidermal growth factor protein, or the activity of basic fibroblast growth factor and/or heparin binding epidermal growth factor. In specific embodiments, synthesis of IGF I, IGF II, basic fibroblast growth factor and/or heparin binding epidermal growth factor mRNA is downregulated by antisense nucleic acids.

The present application also discloses methods to inhibiting or preventing uterine leiomyoma proliferation by administering to a subject a compound that modulates Cyr61 binding to integrin receptors.

### BRIEF DESCRIPTION OF THE DRAWINGS

FIGS. 1(A)-(D). Identification of Cyr61 by RADE methodology and confirmation by Northern Analysis. (A) Representative autoradiograph of ³⁵S-radiolabeled cDNAs generated from total RNA extracted from duplicate leiomyoma (L) and matched myometrial (M) tissues (n=4). (B) Representative northern blot of total RNA isolated from leiomyomas (L) and matched myometrial (M) tissues. Arrows indicate the positions of the 2.4 kb major and the 3.5 kb minor Cyr61 transcripts. (C) Membranes reprobed with a 2.0 kb radiolabeled mouse glyeraldehyde phosphate dehydrogenase (GAPDH) to verify equivalent sample loading. (D) Densimetric analysis of Cyr61 mRNA levels utilizing a Molecular Dynamics phosphorimager and image quantitation software. *Significant decrease in levels compared to myometrial controls.

FIGS. 2(A)-(C). Analysis of Cyr61 protein expression in leiomyoma. (A) Representative Western Blot of tissue protein extracts generated from leiomyoma and matched myometrial tissues. (B) Protein blots were subsequently reprobed with an anti-actin monospecific antibody to confirm equivalent protein loading. (C) Cyr61 protein levels quantitated by densitometric analysis utilizing a Biorad molecular imager. Values represent the mean±SD for 10 patients. *Significant decrease in levels compared to myometrial controls.

FIGS. 3(A)-(B). Differential expression of Cyr61 in multiple human tissues (A) and human muscle tissue (B). Arrows indicate molecular weight markers in kb.

FIGS. 4(A)-(F). Suppression of Cyr61 expression in uterine leiomyoma smooth muscle cells as determined by in situ hybridization. Representative dark field (A, B, E and F) and bright field C and D) photomicrographs of the uterine myometrial (A, C and E) and leiomyoma (B, D and F) tissue sections. Arrows denote representative uterine smooth muscle cells that express Cyr61 transcripts (C). Sense radiolabeled cRNA probes gave no signal above background (E and F). Magnification=200× (A, B, E and F) and 630× © and D). Bars=15 μm (A, B, E and F) and 5 μm C and D).

FIGS. 5(A)-(G). Dysregulation of Cyr61 by 17β-estradiol and basic fibroblast growth factor (bFGF) ex vivo in leiomyomas. Figures (A)-(C) represent fresh myometrial and figures (D)-(F) represent leiomyoma tissue specimens were cultured ex vivo either in the presence of ethanol vehicle, 10 nM 17β-estradiol E₂), 10 nM R5020, 10 ng/ml bFGF, a combination of 10 nM E₂ and 10 μM R5020, 11M ICI 182,780, or a combination of 10 nM E₂ and 1 LM ICI 182,780. Membranes were reprobed with a GAPDH cDNA to account for equivalency in sample loading C and F). Arrows indicate the 2.4 kb major Cyr61 and 6.2 kb ERα transcript. (G) represents calculated data. Values represent the mean±SD for 8 patients. *Significant increase in Cyr61 mRNA levels compared to untreated myometrial tissues.

FIG. 6. Nucleic acid sequence of Cyr61.(SEQ ID NO:1)

FIG. 7. Amino acid sequence of Cyr61 encoded by the nucleic acid sequence of FIG. 6. (SEQ ID NO:2; GenBank Accession Number AAB58319)

### DETAILED DESCRIPTION OF THE INVENTION

The present invention describes methods of inhibiting uterine leiomyoma proliferation, preventing uterine leiomyoma formation, diagnosing uterine leiomyomas, and screening for compounds which inhibit or prevent uterine leiomyomas. These methods evaluate or direct steroid and growth factor mediated regulation of Cyr61 transcription and translation and levels of Cyr61 protein in samples of interest. The present invention also advantageously provides for screening assays and kits. The assay system of the invention is suitable for high throughput screening, e.g., screening thousands of compounds per assay.

The present invention also provides Cyr61-specific antibodies, and related methods of using these materials to detect the presence of Cyr61 proteins and in screens for agonists of Cyr61 for uterine leiomyomas.

### General Definitions

The term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. Purity can be evaluated by chromatography, gel electrophoresis, immunoassay, composition analysis, biological assay, and other methods known in the art.

A “sample” refers to a biological material which can be tested for the presence of Cyr61 protein or Cyr61 nucleic acids. Such samples can be obtained from subjects, such as humans and non-human animals, and include tissue, especially uterine glands, biopsies, blood and blood products; plural effusions; cerebrospinal fluid (CSF); ascites fluid; and cell culture.

The term “non-human animals” includes, without limitation, laboratory animals such as mice, rats, rabbits, hamsters, guinea pigs, etc.; domestic animals such as dogs and cats; and, farm animals such as sheep, goats, pigs, horses, and cows.

The term “ability to elicit a response” includes the ability of a ligand to agonize or antagonize receptor activity.

The term “transformed cell” refers to a modified host cell that expresses a functional protein expressed from a vector encoding the protein of interest. Any cell can be used, but preferred cells are mammalian cells.

The term “assay system” is one or more collections of such cells, e.g., in a microwell plate or some other culture system. To permit evaluation of the effects of a test compound on the cells, the number of cells in a single assay system is sufficient to express a detectable amounts of regulated Cyr61 mRNA or protein expression. The methods of the invention are particularly suitable for use in an assay system to test ligands that modulate transcription and translation of the Cyr61 gene.

A “test compound” is any molecule, such as, for example, an estrogen compound, that can be tested for its ability to modulate Cyr61 expression and/or activity.

The term “leiomyomas” refers to benign tumors composed of smooth muscle and fibrous connective tissue. In a specific embodiment, the leiomyoma is uterine leiomyoma. Leiomyomas also may be referred to as fibroid tumors, fibromyomas, fibromas, fibroleiomyomas, fibroids, or myomas. The term “benign” refers to non-cancerous growths.

The term “myometrium” or “myometrial layer” refers to the muscle layer of the uterus.

Descriptions made herein relating to increasing the level of Cyr61 in an organism or cell include, but are not limited to, methods that stimulate transcription of Cyr61 DNA, stimulate translation of Cyr61 protein, stimulate processing of Cyr61 protein, modulate binding of Cyr61 protein to cellular proteins (e.g., integrin receptors), addition of exogenous Cyr61 protein, or addition of vectors comprising nucleic acid sequences that encode Cyr61 protein.

The Cyr61 of the present invention may be isolated, present, or detected in various mammal sources, including mammal, e.g., human, bovine, porcine, canine, and avian. A preferred source of the present invention is human.

The term “inhibiting uterine leiomyoma proliferation” refers to decreasing the rate of leiomyoma growth or fully stopping the growth. In a preferred embodiment, the decrease of leiomyoma growth is at least 20%, preferably at least 40%, and more preferably at least 80%.

The term “uterine leiomyoma preventing level” refers to the amount needed to inhibit formation of uterine leiomyoma in normal myometrial tissue.

The term “level” refers to a total amount per unit (e.g., cell) or a rate of activity.

The use of italics generally indicates a nucleic acid molecule (e.g., Cyr61 cDNA, gene, etc.); normal text generally indicates the polypeptide or protein. Alternatively, whether a nucleic acid molecule or a protein is indicated can be determined by the content.

The term “amplification” of DNA refers to the use of polymerase chain reaction (PCR) to increase the concentration of a particular DNA sequence within a mixture of DNA sequences. For a description of PCR see Saiki et al., Science, 239:487, 1988.

The term “sequence-specific oligonucleotides” refers to related sets of oligonucleotides that can be used to detect allelic variations or mutations in the Cyr61 gene.

The term “nucleic acid molecule” refers to the phosphate ester form of ribonucleosides (RNA molecules) or deoxyribonucleosides (DNA molecules), or any phosphoester analogs, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear (e.g. restriction fragments) or circular DNA molecules, plasmids, and chromosomes. In discussing the structure of particular double-stranded DNA molecules, sequences may be described according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation. Non-limiting examples of molecular biological manipulation include enzymatic phosphorylation, enzymatic de-phophorylation, enzymatic digestion, and ligation.

The terms “polynucleotide” or “nucleotide sequence” is a series of nucleotide bases (also called “nucleotides”) in DNA and RNA, and means any chain of two or more nucleotides. A nucleotide sequence typically carries genetic information, including the information used by cellular machinery to make proteins and enzymes. These terms include double or single stranded genomic and cDNA, RNA, any synthetic and genetically manipulated polynucleotide, and both sense and anti-sense polynucleotide. This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases, for example thio-uracil, thio-guanine and fluoro-uracil.

The polynucleotides may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

The term “host cell” means any cell of any organism that is selected, modified, transformed, grown, or used or manipulated in any way, for the production of a substance by the cell, for example the expression by the cell of a gene or RNA sequence, a protein or an enzyme. Host cells can further be used for screening or other assays, as described infra.

Generally, a DNA sequence having instructions for a particular protein or enzyme is “transcribed” into a corresponding sequence of RNA. The RNA sequence in turn is “translated” into the sequence of amino acids which form the protein or enzyme. An “amino acid sequence” is any chain of two or more amino acids. Each amino acid is represented in DNA or RNA by one or more triplets of nucleotides. Each triplet forms a codon, corresponding to an amino acid. The genetic code has some redundancy, also called degeneracy, meaning that most amino acids have more than one corresponding codon.

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme.

The term “gene”, also called a “structural gene” means a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription.

A “promoter sequence” is a DNA regulatory region capable of binding a secondary molecule which in a cell and initiating transcription of a coding sequence.

A coding sequence is “under the control” or “operatively associated with” of transcriptional and translational control sequences in a cell when RNA polymerase transcribes the coding sequence into mRNA, which is then trans-RNA spliced (if it contains introns) and translated into the protein encoded by the coding sequence.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed” by the cell. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

The term “transfection” means the introduction of a foreign nucleic acid into a cell. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences, such as start, stop, promoter, signal, secretion, or other sequences used by a cell's genetic machinery. The gene or sequence may include nonfunctional sequences or sequences with no known function. A host cell that receives and expresses introduced DNA or RNA has been “transformed” and is a “transformant” or a “clone.” The DNA or RNA introduced to a host cell can come from any source, including cells of the same genus or species as the host cell, or cells of a different genus or species.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Vectors include plasmids, phages, viruses, etc.

A common type of vector is a “plasmid”, which generally is a self-contained molecule of double-stranded DNA, usually of bacterial origin, that can readily accept additional (foreign) DNA and which can readily introduced into a suitable host cell. A plasmid vector often contains coding DNA and promoter DNA and has one or more restriction sites suitable for inserting foreign DNA. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression cassettes.

A “cassefte” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.”

The term “expression system” means a host cell and compatible vector under suitable conditions, e.g. for the expression of a protein coded for by foreign DNA carried by the vector and introduced to the host cell. Common expression systems include E. coli host cells and plasmid vectors, insect host cells and Baculovirus vectors, and mammalian host cells and vectors.

The term “heterologous” refers to a combination of elements not naturally occurring. For example, heterologous DNA refers to DNA not naturally located in the cell, or in a chromosomal site of the cell. Preferably, the heterologous DNA includes a gene foreign to the cell. A heterologous expression regulatory element is a such an element operatively associated with a different gene than the one it is operatively associated with in nature.

The term “autologous” refers a specimen that is derived from the same individual. For example, autologous tissue refers to different tissue specimens that obtained from the same person. In a specific example, suspect uterine leiomyoma and autologous normal myometrium uterine refers to different uterine tissue samples obtained from the same individual.

The terms “mutant” and “mutation” mean any detectable change in genetic material, e.g. DNA, or any process, mechanism, or result of such a change. This includes gene mutations, in which the structure (e.g. DNA sequence) of a gene is altered, any gene or DNA arising from any mutation process, and any expression product (e.g. protein or enzyme) expressed by a modified gene or DNA sequence. The term “variant” may also be used to indicate a modified or altered gene, DNA sequence, enzyme, cell, etc., i.e., any kind of mutant.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, corresponding to a Tₘ (melting temperature) of 55° C., can be used, e.g., 5×SSC, 0.1% SDS, 0.25% milk, and no formamide; or 30% formamide, 5×SSC, 0.5% SDS. Moderate stringency hybridization conditions correspond to a higher Tₘ, e.g., 40% formamide, with 5×or 6×SCC. High stringency hybridization conditions correspond to the highest Tₘ, e.g., 50% formamide, 5×or 6×SCC. SCC is a 0.15M NaCl, 0.015M Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C. In a specific embodiment, “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

The term “oligonucleotide” refers to a nucleic acid, generally of at least 10, preferably at least 15, and more preferably at least 20 nucleotides, preferably no more than 100 nucleotides, that is hybridizable to a genomic DNA molecule, a cDNA molecule, or an mRNA molecule encoding a gene, mRNA, cDNA, or other nucleic acid of interest. Oligonucleotides can be labeled, e.g., with ³²P-nucleotides or nucleotides to which a label, such as biotin, has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of Cyr61, or to detect the presence of nucleic acids encoding Cyr61. Generally, oligonucleotides are prepared synthetically, preferably on a nucleic acid synthesizer.

The present invention provides antisense nucleic acids (including ribozymes), which may be used to inhibit expression of Cyr61 or to localize Cyr61 mRNA or DNA in a cell. An “antisense nucleic acid” is a single stranded nucleic acid molecule or oligonucleotide which, on hybridizing under cytoplasmic conditions with complementary bases in an RNA or DNA molecule, inhibits the latter's role. If the RNA is a messenger RNA transcript, the antisense nucleic acid is a countertranscript or mRNA-interfering complementary nucleic acid. As presently used, “antisense” broadly includes RNA-RNA interactions, RNA-DNA interactions, ribozymes and RNase-H mediated arrest. Antisense nucleic acid molecules can be encoded by a recombinant gene for expression in a cell (e.g., U.S. Pat. Nos. 5,814,500 and 5,811,234), or alternatively they can be prepared synthetically (e.g., U.S. Pat. No. 5,780,607).

### Leiomyomas

As mentioned above, the term “leiomyomas” refers to tumors that are comprised of smooth muscle and fibrous connective tissue. It is proposed that leiomyomas result from somatic mutations of a single cell. Uterine leiomyomas refer to tumors associated with the uterus. Uterine leiomyomas can be classified based on the location of the tumor and the uterine layer that is affected. Location of uterine leiomyomas may be categorized as (a) cervical, (b) isthmic, or (c) corporal. Cervical uterine leiomyomas generally grow towards the vagina and may cause sinusiorragia and infection. Isthmic uterine leiomyomas frequently cause pain and urinary problems. Corporal uterine leiomyomas, the most common location, are frequently asymptomatic. Uterine leiomyomas may affect the (a) subserous, (b) submucous, or (c) intramural uterine layers. Intramural leiomyomas are the most common form of this tumor and occur within the walls of the uterus.

Epidemiological studies indicate that uterine leiomyomas are present in about 30% of women over the age of 30. Most women with leiomyomas are asymptomatic, with only 35-50% of affected patients experiencing problems. Some problems associated with uterine leiomyomas include, but are not limited to, abnormal uterine bleeding, pain, infertility, urinary symptoms, intestinal symptoms, and venous congestion. Rapidly growing leiomyomas may be an indication of transformation of the benign tumor to malignancy.

### Viral and Non-Viral Vectors

Preferred vectors, particularly for cellular assays in vitro and in vivo, are viral vectors, such as lentiviruses, retroviruses, herpes viruses, adenoviruses, adeno-associated viruses, vaccinia virus, baculovirus, and other recombinant viruses with desirable cellular tropism. Thus, a gene encoding a functional or mutant protein or polypeptide domain fragment thereof can be introduced in vivo, ex vivo, or in vitro using a viral vector or through direct introduction of DNA. Expression in targeted tissues can be effected by targeting the transgenic vector to specific cells, such as with a viral vector or a receptor ligand, or by using a tissue-specific promoter, or both. Targeted gene delivery is described in PCT Publication No. WO 95/28494.

Viral vectors commonly used for in vivo or ex vivo targeting and therapy procedures are DNA-based vectors and retroviral vectors. Methods for constructing and using viral vectors are known in the art (see, e.g., Miller and Rosman, BioTechniques, 1992, 7:980-990). Preferably, the viral vectors are replication-defective, that is, they are unable to replicate autonomously in the target cell. Preferably, the replication defective virus is a minimal virus, i.e., it retains only the sequences of its genome which are necessary for encapsulating the genome to produce viral particles.

DNA viral vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV1) vector (Kaplitt et al., Molec. Cell. Neurosci., 1991, 2:320-330), defective herpes virus vector lacking a glyco-protein L gene, or other defective herpes virus vectors (PCT Publication Nos. WO 94/21807 and WO 92/05263); an attenuated adenovirus vector, such as the vector described by Stratford-Perricaudet et al. (J. Clin. Invest., 1992, 90:626-630; see also La Salle et al., Science, 1993, 259:988-990); and a defective adeno-associated virus vector (Samulski et al., J. Virol., 1987, 61:3096-3101; Samulski et al., J. Virol., 1989, 63:3822-3828; Lebkowski et al., Mol. Cell. Biol., 1988, 8:3988-3996).

Various companies produce viral vectors commercially, including, but not limited to, Avigen, Inc. (Alameda, Calif.; AAV vectors), Cell Genesys (Foster City, Calif.; retroviral, adenoviral, AAV vectors, and lentiviral vectors), Clontech (retroviral and baculoviral vectors), Genovo, Inc. (Sharon Hill, Pa.; adenoviral and AAV vectors), Genvec (adenoviral vectors), IntroGene (Leiden, Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), and Transgene (Strasbourg, France; adenoviral, vaccinia, retroviral, and lentiviral vectors).

Adenovirus vectors. Adenoviruses are eukaryotic DNA viruses that can be modified to efficiently deliver a nucleic acid of the invention to a variety of cell types. Various serotypes of adenovirus exist. Of these serotypes, preference is given, within the scope of the present invention, to using type 2 or type 5 human adenoviruses (Ad 2 or Ad 5) or adenoviruses of animal origin (see PCT Publication No. WO 94/26914). Those adenoviruses of animal origin which can be used within the scope of the present invention include adenoviruses of canine, bovine, murine (example: Mav1, Beard et al., Virology, 1990, 75-81), ovine, porcine, avian, and simian (example: SAV) origin. Preferably, the adenovirus of animal origin is a canine adenovirus, more preferably a CAV2 adenovirus (e.g., Manhattan or A26/61 strain, ATCC VR-800, for example). Various replication defective adenovirus and minimum adenovirus vectors have been described (PCT Publication Nos. WO 94/26914, WO 95/02697, WO 94/28938, WO 94/28152, WO 94/12649, WO 95/02697, WO 96/22378). The replication defective recombinant adenoviruses according to the invention can be prepared by any technique known to the person skilled in the art (Levrero et al., Gene, 1991, 101:195; European Publication No. EP 185 573; Graham, EMBO J., 1984, 3:2917; Graham et al., J. Gen. Virol., 1977, 36:59). Recombinant adenoviruses are recovered and purified using standard molecular biological techniques, which are well known to one of ordinary skill in the art.

Adeno-associated viruses. The adeno-associated viruses (AAV) are DNA viruses of relatively small size that can integrate, in a stable and site-specific manner, into the genome of the cells which they infect. They are able to infect a wide spectrum of cells without inducing any effects on cellular growth, morphology or differentiation, and they do not appear to be involved in human pathologies. The AAV genome has been cloned, sequenced and characterized. The use of vectors derived from the AAVs for transferring genes in vitro and in vitro has been described (see, PCT Publication Nos. WO 91/18088 and WO 93/09239; U.S. Pat. Nos. 4,797,368 and 5,139,941; European Publication No. EP 488 528). The replication defective recombinant AAVs according to the invention can be prepared by cotransfecting a plasmid containing the nucleic acid sequence of interest flanked by two AAV inverted terminal repeat (ITR) regions, and a plasmid carrying the AAV encapsidation genes (rep and cap genes), into a cell line which is infected with a human helper virus (for example an adenovirus). The AAV recombinants which are produced are then purified by standard techniques.

Retrovirus vectors. In another embodiment the gene can be introduced in a retroviral vector, e.g., as described in U.S. Pat. No. 5,399,346; Mann et al., Cell, 1983, 33:153; U.S. Pat. Nos. 4,650,764 and 4,980,289; Markowitz et al., J. Virol., 1988, 62:1120; U.S. Pat. No. 5,124,263; European Publication Nos. EP 453 242 and EP178 220; Bernstein et al., Genet. Eng., 1985, 7:235; McConnick, BioTechnology, 1985, 3:689; PCT Publication No. WO 95/07358; and Kuo et al., Blood, 1993, 82:845. The retroviruses are integrating viruses that infect dividing cells. The retrovirus genome includes two LTRs, an encapsidation sequence and three coding regions (gag, pol and env). In recombinant retroviral vectors, the gag, pol and env genes are generally deleted, in whole or in part, and replaced with a heterologous nucleic acid sequence of interest. These vectors can be constructed from different types of retrovirus, such as, HIV, MoMuLV (“murine Moloney leukaemia virus” MSV (“murine Moloney sarcoma virus”), HaSV (“Harvey sarcoma virus”); SNV (“spleen necrosis virus”); RSV (“Rous sarcoma virus”) and Friend virus. Suitable packaging cell lines have been described in the prior art, in particular the cell line PA317 (U.S. Pat. No. 4,861,719); the PsiCRIP cell line (PCT Publication No. WO 90/02806) and the GP+envAm-12 cell line (PCT Publication No. WO 89/07150). In addition, the recombinant retroviral vectors can contain modifications within the LTRs for suppressing transcriptional activity as well as extensive encapsidation sequences which may include a part of the gag gene (Bender et al., J. Virol., 1987, 61:1639). Recombinant retroviral vectors are purified by standard techniques know to those having ordinary skill in the art.

Retroviral vectors can be constructed to function as infectious particles or to undergo a single round of transfection. In the former case, the virus is modified to retain all of its genes except for those responsible for oncogenic transformation properties, and to express the heterologous gene. Non-infectious viral vectors are manipulated to destroy the viral packaging signal, but retain the structural genes required to package the co-introduced virus engineered to contain the heterologous gene and the packaging signals. Thus, the viral particles that are produced are not capable of producing additional virus.

Retrovirus vectors can also be introduced by DNA viruses, which permits one cycle of retroviral replication and amplifies tranfection efficiency (see PCT Publication Nos. WO 95/22617, WO 95/26411, WO 96/39036 and WO 97/19182).

Lentivirus vectors. In another embodiment, lentiviral vectors can be used as agents for the direct delivery and sustained expression of a transgene in several tissue types, including brain, retina, muscle, liver and blood. The vectors can efficiently transduce dividing and nondividing cells in these tissues, and maintain long-term expression of the gene of interest. For a review, see, Naldini, Curr. Opin. Bioteclinol., 1998, 9:457-63; see also Zufferey, et al., J. Virol., 1998, 72:9873-80). Lentiviral packaging cell lines are available and known generally in the art. They facilitate the production of high-titer lentivirus vectors for gene therapy. An example is a tetracycline-inducible VSV-G pseudotyped lentivirus packaging cell line that can generate virusparticles at titers greater than 106 IU/ml for at least 3 to 4 days (Kafri, et al., J. Virol., 1999, 73: 576-584). The vector produced by the inducible cell line can be concentrated as needed for efficiently transducing non-dividing cells in vitro and in vivo.

Non-viral vectors. In another embodiment, the vector can be introduced in vivo by lipofection, as naked DNA, or with other transfection facilitating agents peptides, polymers, etc.). Synthetic cationic lipids can be used to prepare liposomes for in vivo transfection of a gene encoding a marker (Felgner, et. al., Proc. Natl. Acad. Sci. U.S.A., 1987, 84:7413-7417; Felgner and Ringold, Science, 1989, 337:387-388; see Mackey, et al., Proc. Natl. Acad. Sci. U.S.A., 1988, 85:8027-8031; Ulmer et al., Science, 1993, 259:1745-1748). Useful lipid compounds and compositions for transfer of nucleic acids are described in PCT Patent Publication Nos. WO 95/18863 and WO 96/17823, and in U.S. Pat. No. 5,459,127. Lipids may be chemically coupled to other molecules for the purpose of targeting (see Mackey, et. al., supra). Targeted peptides, e.g., hormones or neurotransmitters, and proteins such as antibodies, or non-peptide molecules could be coupled to liposomes chemically.

Other molecules are also useful for facilitating transfection of a nucleic acid in vivo, such as a cationic oligopeptide (e.g., PCT Patent Publication No. WO 95/21931), peptides derived from DNA binding proteins (e.g., PCT Patent Publication No. WO 96/25508), or a cationic polymer (e.g., PCT Patent Publication No. WO 95/21931).

It is also possible to introduce the vector in vivo as a naked DNA plasmid. Naked DNA vectors for gene therapy can be introduced into the desired host cells by methods known in the art, e.g., electroporation, microinjection, cell fusion, DEAE dextran, calcium phosphate precipitation, use of a gene gun, or use of a DNA vector transporter (see, e.g., Wu et al., J. Biol. Chem., 1992, 267:963-967; Wu and Wu, J. Biol. Chem., 1988, 263:14621-14624; Canadian Patent Application No. 2,012,311; Williams et al., Proc. Natl. Acad. Sci. USA, 1991, 88:2726-2730). Receptor-mediated DNA delivery approaches can also be used (Curiel et al., Hum. Gene Ther., 1992, 3:147-154; Wu and Wu, J. Biol. Chem., 1987, 262:4429-4432). U.S. Pat. Nos. 5,580,859 and 5,589,466 disclose delivery of exogenous DNA sequences, free of transfection facilitating agents, in a mammal. Recently, a relatively low voltage, high efficiency in vivo DNA transfer technique, termed electrotransfer, has been described (Mir et al., C.P. Acad. Sci., 1988, 321:893; PCT Publication Nos. WO 99/01157; WO 99/01158; WO 99/01175).

### Antibodies and Antisense Constructs

The present invention describes antibodies that may be used to detect the presence of Cyr61 in cells and specifically in leiomyoma cells such as uterine leiomyomas. Additionally, the antibodies (e.g., anti-idiotypic antibodies) may be used to inhibit proliferation or prevent formation of uterine leiomyomas. Antibodies used in treatment regimens may be conjugated to pharmaceutically active compounds.

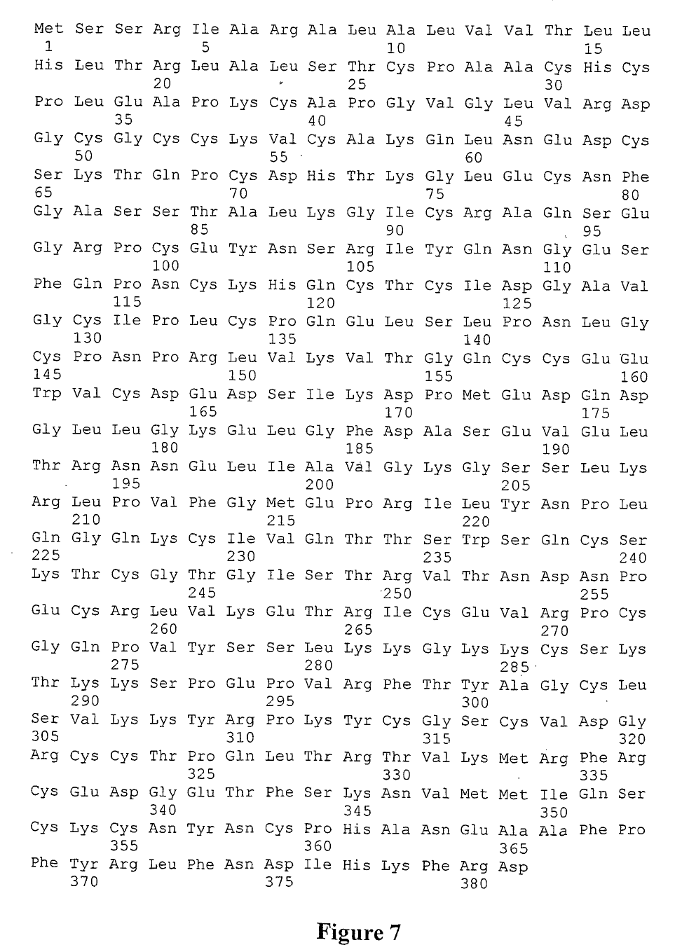

According to the invention, Cyr61 polypeptides produced recombinantly or by chemical synthesis, and fragments or other derivatives, may be used as an imununogen to generate antibodies that recognize the Cyr61 polypeptide or portions thereof. The portion of the polypeptide used as an immunogen may be specifically selected to modulate immunogenicity of the developed antibody. Such antibodies include, but are not limited to, polyclonal, monoclonal, humanized, chimeric, single chain, Fab fragments, and an Fab expression library. An antibody that is specific for human Cyr61 may recognize a wild-type or mutant form of Cyr61. Preferably, the antibody does not recognize or bind to a protein that belongs to the same protein family as Cyr61. In a specific embodiment, the antibody is comprised of at least 8 amino acids, preferably from 8-10 amino acids, and more preferably from 15-30 amino acids. Preferred antibodies are produced to, but not limited to, the amino acids 371-381 of Cyr61 (as depicted in FIG. 7). Preferably, the antibody recognizes or binds amino acids on the Cyr61 polypeptide that are consecutive.

Various procedures known in the art may be used for the production of polyclonal antibodies to polypeptides, derivatives, or analogs. For the production of antibody, various host animals, including but not limited to rabbits, mice, rats, sheep, goats, etc, can be immunized by injection with the polypeptide or a derivative (e.g., fragment or fusion protein). The polypeptide or fragment thereof can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, KLH, dinitrophenol, and potentially useful human adjuvants such as BCG (bacille Calmette-Guerin) and Corynebacterium parvum.

Monoclonal antibodies directed toward a Cyr61 polypeptide, fragment, analog, or derivative thereof, may be prepared by any technique that provides for the production of antibody molecules by continuous cell lines in culture may be used. These include but are not limited to the hybridoma technique originally developed by Kohler and Milstein (Nature 256:495-497, 1975), as well as the trioma technique, the human B-cell hybridoma technique (Kozbor et al., Immunology Today 4:72, 1983; Cote et al., Proc. Natl. Acad. Sci. U.S.A. 80:2026-2030, 1983), and the EBV-hybridoma technique to produce human monoclonal antibodies (Cole et al, in Monoclonal Antibodies and Cancer Therapy, Alan R. Liss, Inc., pp. 77-96, 1985). “Chimeric antibodies” may be produced (Morrison et al., J. Bacteriol. 159:870, 1984; Neuberger et al., Nature 312:604-608, 1984; Takeda et al., Nature 314:452-454, 1985) by splicing the genes from a non-human antibody molecule specific for a polypeptide together with genes from a human antibody molecule of appropriate biological activity.

In the production and use of antibodies, screening for or testing with the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc.

The foregoing antibodies can be used in methods known in the art relating to the localization and activity of the polypeptide, e.g., for Western blotting, imaging the polypeptide in situ, measuring levels thereof in appropriate physiological samples, etc. using any of the detection techniques mentioned above or known in the art. Such antibodies can also be used in assays for ligand binding, e.g., as described in U.S. Pat. No. 5,679,582. Antibody binding generally occurs most readily under physiological conditions, e.g., pH of between about 7 and 8, and physiological ionic strength. The presence of a carrier protein in the buffer solutions stabilizes the assays. While there is some tolerance of perturbation of optimal conditions, e.g., increasing or decreasing ionic strength, temperature, or pH, or adding detergents or chaotropic salts, such perturbations will decrease binding stability.

In a specific Embodiment, antibodies that agonize the activity of Cyr61 polypeptide can be generated. In particular, intracellular single chain Fv antibodies can be used to regulate Cyr61. Such antibodies can be tested using the assays described below for identifying ligands.

In another specific embodiment, the antibodies of the present invention are anti-idiotypic antibodies. These antibodies recognize and or bind to other antibodies present in the system. The anti-idiotypic antibodies may be monoclonal, polyclonal, chimeric, humanized. Additionally, the antibodies may be conjugated to a pharmaceutically active compound. In a specific embodiment, the pharmaceutically active compound is calicheamicin.

In another specific embodiment, antibodies such as, but not limited to, anti-idiotypic, are conjugated to a secondary component, such as, for example, a small molecule, polypeptide, or polynucleotide. The conjugation may be produced through a chemical modification of the antibody, which conjugates the antibody to the secondary component. The conjugated antibody will allow for targeting of the secondary component, such as, for example, an antibiotic to the site of interest. The secondary component may be of any size or length. In a specific embodiment, the secondary component is a pharmaceutically active compound. The pharmaceutically active compound can be, but is not limited to, an anti-leiomyoma agent or calicheamicin.

A further aspect of this invention relates to the use of antibodies, as discussed supra, for targeting a pharmaceutical compound or a Cyr61 peptide. In this embodiment, antibodies against Cyr61 are used to present specific compounds to tumorous cells. The compounds, preferably an anti-tumor agent, when conjugated to the antibodies are referred to as targeted compounds or targeted agents. Methods for generating such target compounds and agents are known in the art. Exemplary publications on target compounds and their preparation are set forth in U.S. Pat. Nos. 5,053,934; 5,773,001; and 6,015,562.

Any desired agent having activity against cancer cells may be employed in generating the targeted agent. Examples of such compounds are discussed in U.S. Pat. No. 6,015,562. See specifically U.S. Pat. Nos. 4,971,198; 5,079,233; 4,539,203; 4,554,162; 4,675,187; and 4,837,206. These publications refer to anti-tumor agents and antibiotics which may be used as the pharmaceutical compound of the target.

The present invention provides antisense nucleic acids (including ribozymes), which may be used to inhibit or prevent expression of Cyr61 by repressing proteins, particularly proteins that suppress Cyr61 effects on cell proliferation. Antisense nucleic acids that increase the total level of Cyr61 also may be used to modulate binding of Cyr61 to intracellular proteins (e.g., integrin receptors). Additionally, antisense nucleic acids also may be used as a diagnostic tool to determine alterations in Cyr61 transcription and/or translation in sample's that are suspected of comprising uterine leiomyomas.

An “antisense nucleic acid” is a single stranded nucleic acid molecule or oligonucleotide which, on hybridizing under cytoplasmic conditions with complementary bases in an RNA or DNA molecule, inhibits the latter's role. In a preferred embodiment, the antisense nucleic acid is at least about 10 nucleotides; preferably at least about 15 nucleotides; and more preferably the length is at least about 20 nucleotides. If the RNA is a messenger RNA transcript, the antisense nucleic acid is a countertranscript or mRNA-interfering complementary nucleic acid. As presently used, “antisense” broadly includes RNA-RNA interactions, RNA-DNA interactions, ribozymes and RNase-H mediated arrest. Antisense nucleic acid molecules can be encoded, by a recombinant gene for expression in a cell (e.g., U.S. Pat. Nos. 5,814,500 and 5,811,234), or alternatively they can be prepared synthetically (e.g., U.S. Pat. No. 5,780,607). Also contemplated are vectors which include these oligonucleotides or anti-sense constructs.

### Compounds

### Ovarian Steroids

An “ovarian steroid” refers to a class of hormonal substances that are secreted from the reproductive organs, specifically the ovaries, including, but not limited to, estrogen and progesterone.

Estrogen compounds are described, for example, in the 1th edition of “Steroids” from Steraloids Inc., Wilton N. H. Non-steroidal estrogens described therein are included, as well. Other compounds included are derivatives, metabolites, and precursors.

Also included are mixtures of more than one compound. Examples of such mixtures are provided in Table II of U.S. Pat. No. 5,554,601 (see column 6). Examples of estrogens either alone or in combination with other agents are provided, e.g., in U.S. Pat. No. 5,554,601.

β-estrogen is the β-isomer of estrogen compounds. α-estrogen is the α-isomer of estrogen components. The term “estradiol” is either α- or β-estradiol unless specifically identified.

The term “E2” is synonymous with 17β-estradiol.

Progesterone compounds are described, for example, in the 9^{th} edition of “The Pharmacological Basis of Therapeutics” from McGraw-Hill, New York, N.Y. Progestin compounds, for example, include progestins containing the 21-carbon skeleton and the 19-carbon (19-nortestosterone) skeleton. Non-steroidal progestin compounds, derivatives, precursors, and metabolites are also contemplated herein.

Preferably, a non-feminizing estrogen compound is used herein. Non-feminizing estrogen compounds refers to compounds that do not produce effects that cause a subject to take on feminine characters. Such a compound has the advantage of not causing uterine hypertrophy and other undesirable side effects, and thus, can be used at a higher effective dosage. Examples of non-feminizing estrogen include Raloxifene (Evista; Eli Lilly), Tamoxifen (Nolvadex; Astra Zeneca), and other selective estrogen receptor modulators.

### Growth factors

Growth factors are a class of proteins that are involved in stimulation of cell division. These proteins interact with cell surface receptors to induce transcription factors to promote cell survival. Growth factor receptors signal through the Ras pathway, a highly conserved signal transduction pathway. The Ras pathway functions to promote cell survival in radiation therapy, and genetic changes in this pathway which produce constitutively activate intracellular survival pathways are often associated with the development of cancer.

Growth factors also include, for example, small molecule compounds that interact with growth factor receptors to produce the same effects as observed with growth factor peptides. Other compounds included are derivatives, metabolites, and precursors of endogenous growth factors. In specific embodiments of the present invention, specific growth factors that are used include, but are not limited to, epidermal growth factor, heparin binding epidermal growth factor, and basic fibroblastic growth factor.

### Assay System

Any cell assay system that allows for assessing functional activities of Cyr61 agonists or antagonists, steroid, non-steroid, and growth factor receptor agonists and antagonists is contemplated by the present invention. In a specific embodiment, the assay can be used to identify compounds that interact with specific isoforms of the steroid receptor to regulate Cyr761 transcription and translation, which can be evaluated by assessing the effects of a test compound, which modulates Cyr61 mRNA transcription, Cyr61 translation, or Cyr61 activity.

Any convenient method permits detection of the expressed product. For example, the invention encompasses Northern blot analysis for detecting Cyr61 mRNA product. The methods comprise, for example, the steps of fractionating total cellular RNA on an agarose gel, transferring RNA to a solid support membrane, and detecting a DNA-RNA complex with a labeled DNA probe, wherein the DNA probe is specific for a particular nucleic acid sequence of Cyr61 under conditions in which a stable complex can form between the DNA probe and RNA components in the sample. Such complexes may be detected by using any suitable means known in the art, such as, for example, ECL and fluorescence, wherein the detection of a complex indicates the presence of C61 in the sample.

Typically, immunoassays use either a labeled antibody or a labeled antigenic component (e.g., that competes with the antigen in the sample for binding to the antibody). Suitable labels include without limitation enzyme-based, fluorescent, chemiluminescent, radioactive, or dye molecules. Assays that amplify the signals from the probe are also known, such as, for example, those that utilize biotin and avidin, and enzyme-labeled immunoassays, such as ELISA assays.

### In Vitro Screening Methods

Candidate agents are added to in vitro cell cultures of host cells, prepared by known methods in the art, and the level of Cyr61 mRNA and/or protein is measured. Various in vitro systems can be used to analyze the effects of a new compound on Cyr61 transcription and translation. Preferably, each experiment is performed more than once, such as, for example, in triplicate at multiple different dilutions of compound.

The host cell screening system of the invention permits two kinds of assays:

direct activation assays (agonist screen) and inhibition assays (antagonist screen). An agonist screen involves detecting changes in the level of expression of the gene by the host cell contacted with a test compound; generally, gene expression increases. If the Cyr61 gene is expressed, the test compound has stimulated Cyr61 transcription via receptor interaction.

An antagonist screen involves detecting expression of the reporter gene by the host cell when contacted with a compound that regulates expression of Cyr61. If Cyr61 expression is decreased, the test compound is a candidate antagonist. If there is no change or an increase in expression of the reporter gene, the test compound is not an effective antagonist.

The assay system described here also may be used in a high-throughput primary screen for agonists and antagonists, or it may be used as a secondary functional screen for candidate compounds identified by a different primary screen, e.g., a binding assay screen that identifies compounds that interact with the receptor and affect Cyr61 transcription.

### In Vivo Testing Using Transgenic Animals

Transgenic animals, and preferably mammals, can be prepared for evaluating the molecular mechanisms of Cyr61. Preferably, for evaluating compounds for use in human therapy, the animals are “humanized” with respect to Cyr61. Such mammals provide excellent models for screening or testing drug candidates. The term “transgenic” usually refers to animal whose germ line and somatic cells contain the transgene of interest, i.e., Cyr61. However, transient transgenic animals can be created by the ex vivo or in vivo introduction of an expression vector of the invention. Both types of “transgenic” animals are contemplated for use in the present invention, e.g., to evaluate the effect of a test compound on Cyr61 or Cyr61 activity.

Thus, human Cyr61, “knock-in” mammals can be prepared for evaluating the molecular biology of this system in greater detail than is possible with human subjects. It is also possible to evaluate compounds or diseases on “knockout” animals, e.g., to identify a compound that can compensate for a defect in Cyr61 or Cyr61 activity. Both technologies permit manipulation of single units of genetic information in their natural position in a cell genome and to examine the results of that manipulation in the background of a terminally differentiated organism.

Although rats and mice, as well as rabbits, are most frequently employed as transgenic animals, particularly for laboratory studies of protein function and gene regulation in vivo, any animal can be employed in the practice of the invention.

A “knock-in” mammal is a mammal in which an endogenous gene is substituted with a heterologous gene (Roemer et al., New Biol. 3:331, 1991). Preferably, the heterologous gene or regulation system is “knocked-in” to a locus of interest, either the subject of evaluation (in which case the gene may be a reporter gene; see Elefanty et al., Proc Natl Acad Sci USA 95:11897,1998) of expression or function of a homologous gene, thereby linking the heterologous gene expression to transcription from the appropriate promoter. This can be achieved by homologous recombination, transposon (Westphal and Leder, Curr Biol 7:530, 1997), using mutant recombination sites (Araki et al., Nucleic Acids Res 25:868, 1997) or PCR (Zhang and Henderson, Biotechniques 25:784, 1998). See also, Coffman, Semin. Nephrol. 17:404, 1997; Esther et al., Lab. Invest. 74:953, 1996; Murakami et al., Blood Press. Suppl. 2:36, 1996.

A “knockout mammal” is an mammal (e.g., mouse) that contains within its genome a specific gene that has been inactivated by the method of gene targeting (see, e.g., U.S. Pat. Nos. 5,777,195 and 5,616,491). A knockout mammal includes both a heterozygote knockout (i.e., one defective allele and one wild-type allele) and a homozygous mutant. Preparation of a knockout mammal requires first introducing a nucleic acid construct that will be used to suppress expression of a particular gene into an undifferentiated cell type termed an embryonic stem cell. This cell is then injected into a mammalian embryo. A mammalian embryo with an integrated cell is then implanted into a foster mother for the duration of gestation. Zhou, et al (Genes and Development, 9:2623-34, 1995) describes PPCA knock-out mice.

The term “knockout” refers to partial or complete suppression of the expression of at least a portion of a protein encoded by an endogenous DNA sequence in a cell. The term “knockout construct” refers to a nucleic acid sequence that is designed to decrease or suppress expression of a protein encoded by endogenous DNA sequences in a cell. The nucleic acid sequence used as the knockout construct is typically comprised of (1) DNA from some portion of the gene (exon sequence, intron sequence, and/or promoter sequence) to be suppressed and (2) a marker sequence used to detect the presence of the knockout construct in the cell. The knockout construct is inserted into a cell, and integrates with the genomic DNA of the cell in such a position so as to prevent or interrupt transcription of the native DNA sequence. Such insertion usually occurs by homologous recombination (i.e., regions of the knockout construct that are homologous to endogenous DNA sequences hybridize to each other when the knockout construct is inserted into the cell and recombine so that the knockout construct is incorporated into the corresponding position of the endogenous DNA). The knockout construct nucleic acid sequence may comprise (1) a full or partial sequence of one or more exons and/or introns of the gene to be suppressed, (2) a full or partial promoter sequence of the gene to be suppressed, or (3) combinations thereof. Typically, the knockout construct is inserted into an embryonic stem cell (ES cell) and is integrated into the ES cell genomic DNA, usually by the process of homologous recombination. This ES cell is then injected into, and integrates with, the developing embryo. However, the invention does not require any particular method for preparing a transgenic animal.

Generally, for homologous recombination, the DNA will be at least about 1 kilobase (kb) in length and preferably 3-4 kb in length, thereby providing sufficient complementary sequence for recombination when the construct is introduced. Transgenic constructs can be introduced into the genomic DNA of the ES cells, into the male pronucleus of a fertilized oocyte by microinjeciton, or by any methods known in the art, e.g., as described in U.S. Pat. Nos. 4,736,866 and 4,870,009, and by Hogan et al., Transgenic Animals: A Laboratory Manual, 1986, Cold Spring Harbor. A transgenic founder animal can be used to breed other transgenic animals; alternatively, a transgenic founder may be cloned to produce other transgenic animals.

Included within the scope of this invention is a mammal in which two or more genes have been knocked out or knocked in, or both. Such mammals can be generated by repeating the procedures set forth herein for generating each knockout construct, or by breeding to mammals, each with a single gene knocked out, to each other, and screening for those with the double knockout genotype.

Regulated knockout animals can be prepared using various systems, such as the tet-repressor system (see U.S. Pat. No. 5,654,168) or the Cre-Lox system (see U.S. Pat. Nos. 4,959,317 and 5,801,030).

### Cloning and Expression of Cyr61

The present invention contemplates analysis and isolation any antigenic fragments of Cyr61 from any source, preferably human. It further contemplates expression of functional or mutant Cyr61 protein for evaluation, diagnosis, or therapy.

Conventional molecular biology, microbiology, and recombinant DNA techniques within the skill of the art may be employed in the use of this invention. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning. A Practical Approach Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

### Methods of Inhibiting Uterine Leiomyoma Proliferation

According to the present invention, upregulation of Cyr61 mRNA or protein can be used to inhibit the proliferation of a Cyr61 associated disease, such as uterine leiomyomas. The present invention provides for methods that inhibit proliferation of uterine leiomyomas by administering to a subject a therapeutically effective amount of a compound that stimulates the synthesis of mRNA encoding Cyr61, the translation of Cyr61 mRNA, the expression of Cyr61 protein, or the activity of Cyr61 in leiomyoma tissues. The present invention further provides for methods that inhibit proliferation of uterine leiomyomas by increasing the total level of Cyr61 protein in the cell. These methods include, but are not limited to, delivery of the Cyr61 protein to the cell, administration of an expression vector encoding the Cyr61 protein, and administration of a therapeutically effective amount of a compound that modulates binding of Cyr61 to intracellular proteins (e.g., integrin receptors). The compound may be formulated into a pharmaceutical composition (described below) for administration to the subject. The present invention provides for methods that inhibit proliferation of uterine leiomyomas by administering to a subject a therapeutically effective amount of recombinant DNA to stimulate Cyr61 protein expression or recombinant Cyr61 protein. In a specific embodiment, inhibition of the proliferation of uterine leiomyoma is observed when proliferation is decreased by at least 20%, preferably by at least 40%, and more preferably by at least 80%.

Levels of IGF I, IGF II, bFGF, and FIB-EGF are upregulated in leiomyomas, such as, for example, uterine leiomyomas. The decrease of Cyr61 in leiomyomas may augment the activity of IGFs and growth factors, and thus these molecules may be more effective in stimulating cell proliferation. Therefore, a further embodiment of the present invention contemplates methods for stimulates the synthesis of mRNA encoding Cyr61, the translation of Cyr61 mRNA, the expression of Cyr61 protein, or the activity of Cyr61 and decreasing the synthesis of mRNA, the translation of mRNA, the expression of, or the activity of IGF I, IGF II, bFGF, HB-EGF, or any combination thereof.

The effective amounts of the compounds of the present invention may vary according to a variety of factors such as the individual's condition, weight, sex and age and the mode of administration. This amount of a compound can be determined experimentally by methods well-known in the art such as by establishing a matrix of dosages and frequencies and assigning a group of experimental subjects to each point in the matrix.

### Methods of Preventing Uterine Leiomyoma Formation

According to the present invention, Cyr61 protein expression is higher in autologous myometrial tissue compared to uterine leiomyoma tissue. The present invention provides for methods that prevent formation of uterine leiomyomas by administering to a subject a therapeutically effective amount of a compound that stimulates the synthesis of mRNA encoding Cyr61, the translation of Cyr61 mRNA, the expression of Cyr61 protein, or the activity of Cyr61. The present invention further provides for methods that prevent formation of uterine leiomyomas by increasing the total level of Cyr61 protein in the cell. These methods include, but are not limited to, delivery of the Cyr61 protein to the cell, administration of an expression vector encoding the Cyr61 protein, and administration of a therapeutically effective amount of a compound that modulates binding of Cyr61 to intracellular proteins (e.g., integrin receptors). The compound may be formulated into a pharmaceutical composition (described below) for administration to the subject. The present invention provides for methods that prevent formation of uterine leiomyomas by administering to a subject a therapeutically effective amount of recombinant DNA to stimulate Cyr61 protein expression or recombinant Cyr61 protein.

As previous reports have shown, levels of IGF I, IGF II, bFGF, and HB-EGF are upregulated in leiomyomas, such as, for example, uterine leiomyomas. The decrease of Cyr61 in leiomyomas may augment the activity of IGFs and growth factors, and thus these molecules may be more effective in stimulating cell proliferation. Therefore, a further embodiment of the present invention contemplates methods to stimulate the synthesis of mRNA encoding Cyr61, the translation of Cyr61 mRNA, the expression of Cyr61 protein, or the activity of Cyr61 and/or decreasing the synthesis of mRNA, the translation of mRNA, the expression of, or the activity of IGF I, IGF II, bFGF, HB-EGF, or any combination thereof.

The effective amounts of these compounds may vary according to a variety of factors such as the individual's condition, weight, sex and age and the mode of administration. This amount of test compound can be determined experimentally by methods well-known in the art such as by establishing a matrix of dosages and frequencies and assigning a group of experimental subjects to each point in the matrix.

### Methods of Diagnosis

According to the present invention, decreased levels of Cyr61 mRNA or protein as compared to levels normally expressed in myometrial tissues can be detected to diagnose a Cyr61 associated disease, such as uterine leiomyomas. In the present invention, the level of Cyr61 mRNA or protein in suspect tissue is compared to the level of Cyr61 mRNA or protein present in normal myometrial tissue obtained from the same individual (i.e., autologous myometrial tissue). A lower level of Cyr61 protein and Cyr61 mRNA in the suspect tissue compared to the normal tissue indicates the presence of uterine leiomyoma. Preferably, Cyr61 mRNA level or Cyr61 protein levels in the suspect tissue is equal to or greater than 3 fold lower than in normal tissue. More preferably the level is from about 9 to about 10 fold lower than in normal tissue. Lower levels may be used to develop treatment regimens that also include at least two treatment methods in addition to the Cyr61 related treatments disclosed herein. Levels of Cyr61 mRNA and Cyr61 protein in suspect tissues are compared to normal tissues by normalizing the level of additional mRNAs and proteins (e.g., GAPDH) present in the cells.

The various methods for detecting such decreased levels of Cyr61 mRNA or protein expression include, but are not limited to, Northern blots, in situ hybridization studies, Western blots, ELISA, radioimmunoassay, “sandwich” immunoassays, immunoradiometric assays, gel diffusion precipitation reactions, immunodiffusion assays, in situ immunoassays using colloidal gold, enzyme or radioisotope labels, for example), precipitation reactions, complement fixation assays, immunofluorescence assays, protein A assays, and immunoelectrophoresis assays, etc.

### Nucleic Acid Assays

The DNA may be obtained from any cell source. DNA is extracted from the cell source or body fluid using any of the numerous methods that are known in the art. It will be understood that the particular method used to extract DNA will depend on the nature of the source. Generally, the minimum amount of DNA to be extracted for use in the present invention is about 25 pg (corresponding to about 5 cell equivalents of a genome size of 4×10⁹ base pairs). Sequencing methods are described in detail, supra.

In another alternate embodiment, RNA is isolated from biopsy tissue using methods known to those of ordinary skill in the art such as, for example, guanidium thiocyanate-phenol-chloroform extraction (Chomocyznslci et al., Anal. Biochem., 162:156, 1987). The isolated RNA is then subjected to coupled reverse transcription and amplification by polymerase chain reaction (RT-PCR), using specific oligonucleotide primers that are specific for a selected site. Conditions for primer annealing are chosen to ensure specific reverse transcription and amplification; thus, the appearance of an amplification product is diagnostic of the presence of a particular genetic variation. In another embodiment, RNA is reverse-transcribed and amplified, after which the amplified sequences are identified by, e.g., direct sequencing. In still another embodiment, cDNA obtained from the RNA can be cloned and sequenced to identify a mutation.

### Protein Assays

In an alternate embodiment, tissue is obtained from a subject. Antibodies that are capable of specifically binding to Cyr61 are then contacted with samples of the tissue to determine the presence or absence of a Cyr61 polypeptide specified by the antibody. The antibodies may be polyclonal or monoclonal, but preferably are monoclonal. Measurement of specific antibody binding to cells may be accomplished by any known method, e.g., quantitative flow cytometry, enzyme-linked or fluorescence-linked immunoassay, Western analysis, and the like. Generally, the minimum amount of protein to be extracted, for immunoassays, for use in the present invention is about 20 μg.

Immunoassay technology, e.g., as described in U.S. Pat. Nos. 5,747,274 and 5,744,358, and particularly solid phase “chromatographic” format immunoassays, are preferred for detecting proteins in blood or blood fractions.

### Pharmaceutical Compositions

The test compounds, salts thereof, antibodies, proteins, expression vectors and antisense constructs may be formulated into pharmaceutical compositions. The pharmaceutical composition comprises a therapeutically or stimulating effective amount of at least one of the above. This can be an amount effective to increase Cyr61 expression or activity, transcrption of the Cyr61 gene, or the Cyr61 protein within the targeted cells. Compositions can comprise Cyr61 protein or fragments of the protein. Fragments of the Cyr61 protein will preferably retain the functional activities associated with the full length protein. In a specific embodiment, the C-terminal region of the Cyr61 protein may be altered, deleted, or mutated to increase or decrease Cyr61 protein expression or function.

The pharmaceutical compositions also typically include a pharmaceutically acceptable carrier (or dosing vehicle), such as ethanol, glycerol, water, and the like. Examples of such carriers and methods of formulation are described in Remington's Pharmaceutical Sciences, 18th edition (1990), Mack Publishing Company.

The pharmaceutical composition may also include other additives, such as a flavorant, a sweetener, a preservative, a dye, a binder, a suspending agent, a colorant, a disintegrant, an excipient, a diluent, a lubricant, a plasticizer, or any combination of any of the foregoing. Suitable binders include, but are not limited to, starch; gelatin; natural sugars, such as glucose and beta-lactose; corn sweeteners; natural and synthetic gums, such as acacia, tragacanth, and sodium alginate; carboxymethylcellulose; polyethylene glycol; waxes; and the like. Suitable lubricants include, but are not limited to, sodium oleate, sodium stearate, magnesium stearate, sodium benzoate, sodium acetate, sodium chloride and the like. Suitable disintegrators include, but are not limited to, starch, methyl cellulose, agar, bentonite, xanthan gum and the like.

Suitable salts of the test compounds include, but are not limited to, acid addition salts, such as those made with acids, such as hydrochloric, hydrobromic, hydroiodic, perchloric, sulfuric, nitric, a phosphoric, acetic, propionic, glycolic, lactic pyruvic, malonic, succinic, maleic, fumaric, malic, tartaric, citric, benzoic, carbonic cinnamic, mandelic, methanesulfonic, ethanesulfonic, hydroxyethanesulfonic, benezenesulfonic, p-toluene sulfonic, cyclohexanesulfamic, salicyclic, p-aminosalicylic, 2-phenoxybenzoic, and 2-acetoxybenzoic acid; and salts made with saccharin. Other suitable salts of the compounds include, but are not limited to, alkali metal salts, such as sodium and potassium salts; alkaline earth metal salts such as calcium and magnesium salts; and salts formed with organic ligands, such as quaternary ammonium salts.

Representative salts include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate salts of the compounds of the present invention.

The present invention includes prodrugs of the test compounds. Prodrugs include, but are not limited to, functional derivatives of the test compounds of the present invention which are readily convertible in vivo into the compounds of the present invention. Conventional procedures for the selection and preparation of suitable prodrug derivatives are described, for example, in “Design of Prodrugs”, ed. H. Bundgaard, Elsevier, 1985.

The pharmaceutical compositions may be formulated as unit dosage forms, such as tablets, pills, capsules, boluses, powders, granules, sterile parenteral solutions or suspensions, sterile I.V. solutions, sterile I.M. solutions, sterile intrauterine solutions, elixirs, tinctures, metered aerosol or liquid sprays, drops, ampoules, autoinjector devices or suppositories for oral, parenteral, intranasal, occular, mucosal, transdernal, bucal, topical, sublingual or rectal administration or for administration by inhalation or insufflation, for example. The unit dosage form may be in a form suitable for sustained or delayed release, such as, for example, an insoluble salt of the compound, e.g. a decanoate salt, adapted to provide a depot preparation for intramuscular injection.

Solid unit dosage forms may be prepared by mixing the compound of the present invention with a pharmaceutically acceptable carrier and any other desired additives to form a solid preformulation composition. Examples of suitable additives for solid unit dosage forms include, but are not limited to, starches, such as corn starch; lactose; sucrose; sorbitol; talc; stearic acid; magnesium stearate; dicalcium phosphate; gums, such as vegetable gums; and pharmaceutical diluents, such as water. The solid preformulation composition is typically mixed until a homogeneous mixture of the compound of the present invention and the additives is formed, i.e., until the compound is dispersed evenly throughout the composition, so that the composition may be readily subdivided into equally effective unit dosage forms. The solid preformulation composition is then subdivided into unit dosage forms of the type described above.

Tablets or pills can also be coated or otherwise compounded to form a unit dosage form which has prolonged action, such as time release and sustained release unit dosage forms. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. The compound may be released immediately upon administration or may be formulated such that the compound is released in a sustained manner over a specified time course, such as, for example, 2-12 hours.

Liquid unit dosage forms include, but are not limited to, aqueous solutions, suitably flavored syrups, aqueous or oil suspensions, and flavored emulsions with edible oils, such as cottonseed oil, sesame oil, coconut oil or peanut oil, as well as elixirs and similar pharmaceutical vehicles. Suitable dispersing and suspending agents for aqueous suspensions include, but are not limited to, synthetic and natural gums, such as tragacanth, acacia, alginate, dextran, sodium carboxymethylcellulose, methylcellulose, polyvinyl-pyrrolidone and gelatin.

Suitable pharmaceutically acceptable carriers for topical preparations include, but are not limited to, alcohols, aloe vera gel, allantoin, glycerine, vitamin A and E oils, mineral oil, PPG2 myristyl propionate, and the like. Such topical preparations may be liquid drenches, alcoholic solutions, topical cleansers, cleansing creams, skin gels, skin lotions, and shampoos in cream or gel formulations (including, but not limited to aqueous solutions and suspensions). Typically, these topical preparations contain a suspending agent, such as bentonite, and optionally, an antifoaming agent. Generally, topical preparations contain from about 0.005 to about 10% by weight and preferably from about 0.01 to about 5% by weight of the compound, based upon 100% total weight of the topical preparation.

Pharmaceutical compositions of the present invention for administration parenterally, and in particular by injection, typically include an inert liquid carrier, such as water; vegetable oils, including, but not limited to, peanut oil, cotton seed oil, sesame oil, and the like; and organic solvents, such as solketal, glycerol formal and the like. A preferred liquid carrier is vegetable oil. These pharmaceutical compositions may be prepared by dissolving or suspending the compound of the present invention in the liquid carrier. Generally, the pharmaceutical composition for parenteral administration contains from about 0.005 to about 10% by weight of the compound of the present invention, based upon 100% weight of total pharmaceutical composition.

The compounds of the present invention can also be administered in the form of liposome delivery systems, such as small unilamellar vesicles, large unilamellar vesicles and multilamellar vesicles. Liposomes can be formed from a variety of phospholipids, such as cholesterol, stearylanine or phosphatidylcholines.

Compounds of the present invention may also be delivered by the use of monoclonal antibodies as individual carriers to which the compound molecules are coupled. The compounds of the present invention may also be coupled with soluble polymers as targetable drug carriers. Such polymers include, but are not limited to, polyvinylpyrrolidone, pyran copolymer, polyhydroxypropylmethacrylamidephenol, polyhydroxyethylaspartamidephenol, and polyethyleneoxideopolylysine substituted with palmitoyl residues. Furthermore, the compounds of the present invention may be coupled to biodegradable polymers for controlling the release of the compound, for example, polylactic acid, polyepsilon caprolactone, polyhydroxy•butyric acid, polyorthoesters, polyacetals, polydihydropyrans, polycyanoacrylates and cross-linked or amphipathic block copolymers of hydrogels.

The pharmaceutical compositions of the present invention may be administered to an animal, preferably a human being, in need thereof to stimulate Cyr61 transcription or expression such as, for example, through activation of a steroid or growth factor receptor, or the like.

The effective amounts of the active agents and active metabolites of the active agents of the pharmaceutical composition of the present invention may vary according to a variety of factors such as the individual's condition, weight, sex and age and the mode of administration. This amount of test compound can be determined experimentally by methods well-known in the art such as by establishing a matrix of dosages and frequencies and assigning a group of experimental subjects to each point in the matrix.

The compound of the present invention may be administered alone at appropriate dosages defined by routine testing in order to obtain optimal activity while minimizing any potential toxicity. In addition, co-administration or sequential administration of other active agents may be desirable.

The daily dosage of the compounds of the present invention may be varied over a wide range. For oral administration, the pharmaceutical compositions are preferably provided in the form of scored or unscored tablets for the symptomatic adjustment of the dosage to the patient to be treated. The dosage amount may be adjusted when combined with other active agents as described above to achieve desired effects. On the other hand, unit dosage forms of these various active agents may be independently optimized and combined to achieve a synergistic result wherein the pathology is reduced more than it would be if either active agent were used alone.

Advantageously, the pharmaceutical compositions may be administered in a single daily dose, or the total daily dosage may be administered in divided doses of two, three or four times daily.

For combination treatment with more than one active agent, where the active agents are in separate dosage formulations, the active agents can be administered concurrently, or they each can be administered at separately staggered times.

### Gene Therapy

The lack of Cyr61 expression in leiomyomas may also be due to allelic loss or alterations of chromosome 1p22-p31 in which the gene is located, abrogation of the estrogen or growth factor-signalling pathway, and/or mutations of the ER and growth factor response elements contained within the promoter region. Providing the host with an alternative copy of Cyr61 may overcome any mutations in the gene that may be naturally occurring.

In a specific embodiment, vectors comprising a sequence encoding a Cyr61 of the invention are administered to treat or prevent a disease or disorder associated with the lack of expression of a functional Cyr61 protein or expression of a mutated Cyr61.

Any of the methods for gene therapy available in the art can be used according to the present invention. Exemplary methods are described below.

For general reviews of the methods of gene therapy, see, Goldspiel et al., Clinical Pharmacy, 1993, 12:488-505; Wu and Wu, Biotherapy, 1991, 3:87-95; Tolstoshev, Ann. Rev. Pharmacol. Toxicol., 1993, 32:573-596; Mulligan, Science, 1993, 260:926-932; and Morgan and Anderson, Ann. Rev. Biochem., 1993, 62:191-217; May, TIBTECH, 1993, 11:155-215). Methods commonly known in the art of recombinant DNA technology that can be used are described in Ausubel et al., (eds.), 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al., (eds.), 1994, Current Protocols in Human Genetics, John Wiley & Sons, NY. Vectors suitable for gene therapy are described above.

In one aspect, the therapeutic vector comprises a nucleic acid that expresses Cyr61 in a suitable host. In particular, such a vector has a promoter operationally linked to the coding sequence for Cyr61. The promoter can be inducible or constitutive and, optionally, tissue-specific. In specific embodiments of the present invention, the promoters are the estrogen response element or the fibroblast growth factor response element. In another embodiment, a nucleic acid molecule is used in which the antibody coding sequences and any other desired sequences are flanked by regions that promote homologous recombination at a desired site in the genome, thus providing for intrachromosomal expression of Cyr61 (Koller and Smithies, Proc. Natl. Acad. Sci. USA, 1989, 86:8932-8935; Zijlstra et al., Nature, 1989, 342:435-438).

Delivery of the vector into a patient may be either direct, in which case the patient is directly exposed to the vector or a delivery complex, or indirect, in which case, cells are first transformed with the vector in vitro then transplanted into the patient. These two approaches are known, respectively, as in vivo and ex vivo gene therapy.

In a specific embodiment, the vector is directly administered in vivo, where it enters the cells at the organism and mediates expression of Cyr61. This can be accomplished by any of numerous methods known in the art, e.g., by constructing it as part of an appropriate expression vector and administering it so that it becomes intracellular, e.g., by infection using a defective or attenuated retroviral or other viral vector (see, U.S. Pat. No. 4,980,286), or by direct injection of naked DNA, or by use of microparticle bombardment (e.g., a gene gun; Biolistic, Dupont); or coating with lipids or cell-surface receptors or transfecting agents, encapsulation in biopolymers (e.g., poly-*-1-*4-N-acetylucosamine polysaccharide; see, U.S. Pat. No. 5,635,493), encapsulation in liposomes, microparticles, or microcapsules; by administering it in linkage to a peptide or other ligand known to enter the nucleus; or by administering it in linkage to a ligand subject to receptor-mediated endocytosis (see, e.g., Wu and Wu, J. Biol. Chem., 1987, 62:4429-4432), etc. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publication Nos. WO 92/06180, WO 92/22635, WO 92/20316 and WO 93/14188). Alternatively, the nucleic acid can be introduced intracellularly and incorporated within host cell DNA for expression by homologous recombination (Koller and Smithies, Proc. Natl. Acad. Sci. U.S.A., 1989, 86:8932-8935; Zijlstra, et al., Nature, 1989, 342:435-438). These methods are in addition to those discussed above in conjunction with “Viral and Non-viral Vectors”.

Alternatively, single chain antibodies can also be administered, for example, by expressing nucleotide sequences encoding single-chain antibodies within the target cell population by utilizing, for example, techniques such as those described in Marasco et al. Proc. Natl. Acad. Sci. USA, 1993, 90:7889-7893).

The form and amount of therapeutic nucleic acid envisioned for use depends on the type of disease and the severity of the desired effect, patient state, etc., and can be determined by one skilled in the art.

### EXAMPLES

The present invention will be better understood by reference to the following Examples, which are provided by way of exemplification and not by way of limitation.

Materials Anti-Cyr61 polyclonal antisera were generated at the Louisiana State University Medical Center Core Facilities (Baton Rouge, La.) to amino acids 371-381 (RLFNDIHKFRD; SEQ ID NO:3) of human Cyr61 (FIG. 7; SEQ ID NO:2) protein. A cysteine was added to the N-terminus for coupling to carrier proteins. Peptides were synthesized using an automated phase peptide synthesizer using 9-fluorenylmethyloxycarbonyl (Fmoc) chemistry (PE biosystems 9050+). A Waters Delta Prep 400 preparative chromatography system, with a C18 Phenomenex Jupiter column (250×21.20 mm, 10μ diameter) equipped with a photo diode array detector was used to purify the peptide. A flow rate, through the column, in excess of 100 mL/min purified about 400-500 mgs of peptide. The identity and purity of the antigenic peptide was evaluated using a PE Biosystem DE-MALDI mass spectrometer. Peptide was subsequently coupled to heyhole-limpet hemocyanin and mixed with an equal volume of Complete and Incomplete Feund's Adjuvant.

The mixture was then injected into female New Zealand white rabbits (200 μg antigen and adjuvant mixture/rabbit). On days 14 and 28, rabbits were administered a booster injection that was the same size as the initial injection. On day 38, blood from rabbits was tested using an ELISA (using a Strepavidin/Biotin system) for antibody presence. If an increased antibody titer is required, rabbits were administered a booster injection that was the same sample size as the initial injection on day 42. Serum was collected from the rabbits on day 52 and frozen. Polyclonal antibodies were affinity purified by attaching the antigen to a stationary phase (Sulfo-Link Resin, Pierce) using the side chain of cysteine. Approximately 30 mL of serum was loaded through the column and then washed out to remove non-binding proteins. Antibodies were eluted with 3.5 M MgCl₂/ethyl glycol. Eluted proteins are dialyzed and then concentrated to approximately 1 mg/mL. Concentration is determined by OD at 280 mm.

17β-estradiol (E₂) was purchased from Sigma-Aldrich (St. Louis, Mo.), the progesterone receptor agonist, R5020, was obtained from NEN Life Science Product, Inc. (Boston, Mass.), bFGF was purchased from R & D Systems, Inc. (Minneapolis, Minn.) and the ER antagonist ICI 182,780 was generously provided by Zeneca Pharmaceuticals (Wilmington, Del.).

Study subjects and tissue procurement Uterine leiomyomas and matched myometrial specimens were obtained according to protocols approved by the Institutional Review Boards following routine hysterectomy at the Department of Obstetrics and Gynecology, Pennsylvania Hospital. Tissue samples were provided from patients between the ages of 38 to 53 (median age=45) who were not on hormone replacement therapy nor prescribed gonadotropin releasing hormone agonists (n=38). All but one patient had experienced normal menstrual cycles prior to surgery. Tissue specimens were immediately frozen in liquid nitrogen following hysterectomies for total RNA isolation or fixed in 10% neutral-buffered formalin for in situ hybridization. Tissues for ex vivo culturing were placed in phenol-red free DMEM/Ham's F12 media (Gibco BRL, Rockville, Md.) containing 100 U/ml penicillin, 100 mg/ml streptomycin, and 250 ng/ml amphotericine B as a fungizone and transported on ice.

Identification of regulated genes using rapid analysis of differential expression (RADE). RADE was performed as previously reported (Liang P, Pardee A B. 1997. Methods In Molecular Biology. Humana Press, page 150). Briefly, total RNA isolated from matched leiomyoma and myometrial tissues (n=4) was used for RADE analysis and each RNA sample was analyzed in duplicate. Synthesis of cDNAs was accomplished by using p(dT)₁₈ oligonuceotides ending with either A, G, or C. Following cDNA synthesis, genes were amplified using a combination of random oligomers, appropriate p(dT)₁₈ downstream primers, and ³⁵S labeled dATP. The resulting products were amplified in duplicates, separated on SDS polyacrylamide sequencing gels and detected by autoradiography. After the procedure was repeated, candidate cDNA fragments were extracted from polyacrylamide gel slices and amplified by PCR using the appropriate pair of the primers. Amplified products were resolved by agarose gel electrophoresis, subcloned into pBR322, sequenced using ABI 377/373 sequencers and were analyzed using BLASTN software (Altschul et al. Nucleic Acids Research, 1997, 25:3389-3402).

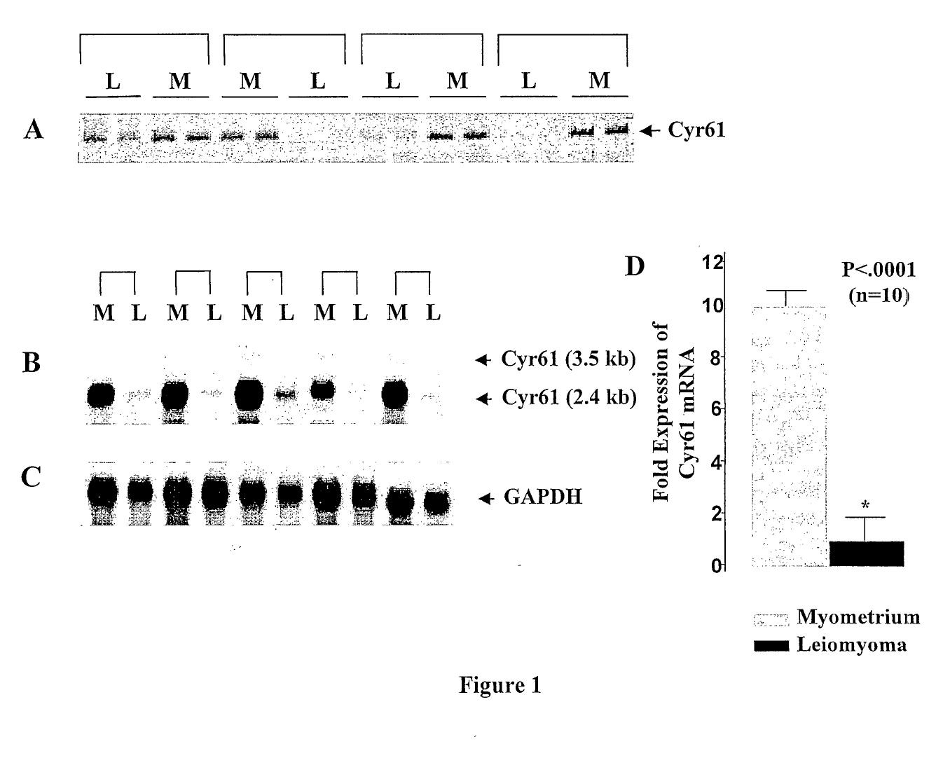

RADE analysis of total RNA demonstrated decreased expression of a 410 nucleotide cDNA fragment in 4 out of 4 leiomyoma specimens compared to matched myometrial controls (FIG. 1A). Sequence analysis using BLASTN software demonstrated that the cDNA fragment was 96% homologous to the C-terminal portion of human Cyr61.

Northern blotting for Cyr61 and ER a Total cellular RNA was isolated from myometrial and leiomyoma tissue homogenates by guanidium isothiocynate lysis followed by phenol/chloroform extraction. Subsequently, total cellular RNA (20 μg) was subjected to electrophoresis in a 1% agarose gel containing 1 M formaldehyde. Separated RNA transcripts were transferred onto nylon membranes by capillary electrophoresis and subsequently prehybridized at 60° C. in RapidHyb hybridization solution (Amersham, Arlington Heights, Ill.). A 0.41 kb human Cyr61 cDNA fragment was radiolabeled with [α-³²P]-dCTP (3,000 Ci/mmol) using the random-primer technique (Rediprime II, Amersham) and used as the hybridization probe. The radiolabeled probe (1×10⁶ cpm/ml) was hybridized to membranes for 4 h at 60° C. Membranes were washed twice in 1×SSPE (0.15 M NaCl, 1 μM EDTA, and 0.01 M sodium phosphate, pH 7.4) and 0.1% SDS for 15 min at 25° C., followed by a final wash in 0.1×SSPE and 0.1% SDS for 5 min at 60° C. For estrogen receptor α (ERα) expression, membranes were reprobed with a 1.96 kb human ERα cDNA (1.96 kb full length coding region) that was radiolabeled with [α-³²P]-dCTP (3,000 Ci/mmol), hybridized and washed as described above. Relative levels of Cyr61 were normalized to glyceraldehyde-3-phosphate dehydrogenase (GAPDH) after reprobing membranes with a ³²P-radiolabeled oligonucleotide according to manufacturers protocol (endlabeling kit, GibcoBRL, Rockville, Md.).

Northern analysis using total RNA isolated from 10 patients was performed. Cyr61 transcripts were markedly diminished in leiomyoma specimens when compared to autologous myometrium in 10 out of 10 patients (FIG. 1B) studied. The decrease in Cyr61 mRNA, normalized to GAPDH mRNA levels, was greater than 9 fold compared to the high basal levels present in autologous myometrium (FIG. 1D).

Protein extraction aid immunoblotting for Cyr61 Tissue protein extracts were prepared from leiomyoma and matched myometrial tissue specimens by homogenization in 50 mM Tris, pH 8.0, 250 mM NaCl, 1.0% Nonidet P-40, 1.0% Trtion-X 100, 2% SDS, 0.5% deoxycholate, 1 mM EDTA, and a protease inhibitor cocktail containing 10 μg/ml pepstatin, aprotinin, and leupeptin (Sigma, St. Louis, Mo.). Protein extracts (20 μg) were subjected to SDS-polyacrylamide gel electrophoresis under reducing conditions in 10% bis-acrylamide and electrophoretically transferred to polyvinyl difluoride membrane (Immobilon-P, Biorad, Redding, Calif.). Membranes were blocked with 5% dry milk on TBS/0.1% Tween-20 (TBST), and incubated with anti-Cyr61 pAb (10 μg/ml). Primary antibody binding was detected using a donkey anti-rabbit IgG antibody conjugated to horseradish peroxidase (HRP) and an enhanced chemiluminescence detection system (Amersham). In order to normalize protein levels, Cyr61 western blots were subsequently reprobed with a pan-actin monoclonal antibody (Sigma) and detected with a donkey anti-mouse secondary antibody conjugated to HRP.

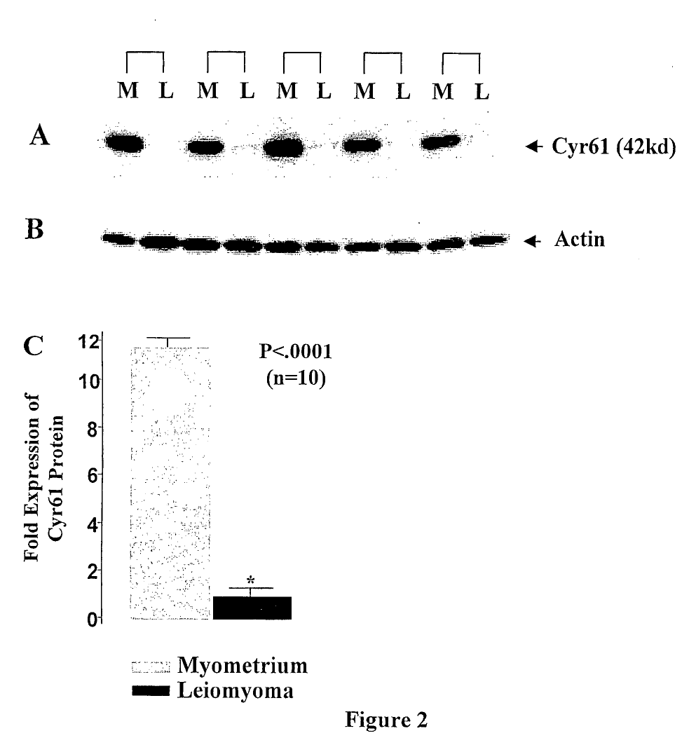

Immunoblot analysis of whole cell lysates generated from leiomyoma and matched myometrial controls demonstrated a greater than 10 fold decrease in Cyr61 protein levels in 10 out of 10 patients studied (FIG. 2A and C).

In situ hybridization For riboprobe synthesis, a 0.28 kb human Cyr61 cDNA fragment was positionally cloned into the EcoRI and Hind III sites of pGEM4Zf plasmid (Promega Corp, Madison, Wis.) to generate pGEM4Zf/Cyr61. Radiolabeled ³⁵S-UTP sense and antisense cRNA transcripts were transcribed in vitro with T3 and T7 RNA polymerases, respectively, using the Gemini Riboprobe system (Promega). In situ hybridization was performed as described previously, using formalin-fixed leiomyoma and matched myometrial specimens. Briefly, processed slides were hybridized overnight with 100-150 μl of an antisense or sense (control) riboprobe at 4.7×10⁶ DPM/slide in 50% formamide hybidization mixture including 5% dextran sulfate and 200 mM dithiothreitol (DTT) at 55° C. in a humidified chamber containing 50% formiamide/600 mM NaCl. Slides were washed three times at room temperature in 2×SSC (0.3 M NaCl, 0.03 M sodium citrate, pH 7.0)/10 mM DTT, followed by RNase A (20 μg/ml) treatment for 30 minutes at 37° C., and washed for 15 min in 0.1×SSC at room temperature. Slides were further washed at 65° C. with 0.1×SSC and dehydrated with a graded series of alcohol:ammonium acetate (70%, 95%, and 100%). Air-dried slides were exposed to X-ray film (Amersham) for 3 days for preliminary examination and then dipped in NTB2 nuclear emulsion (Eastman Kodak, Rochester, N.Y.) diluted 1:1 with 600 mM ammonium acetate. Slides were exposed for 31 days in light-tight, black desiccated boxes, photographically processed, stained in cresyl violet and coverslipped.

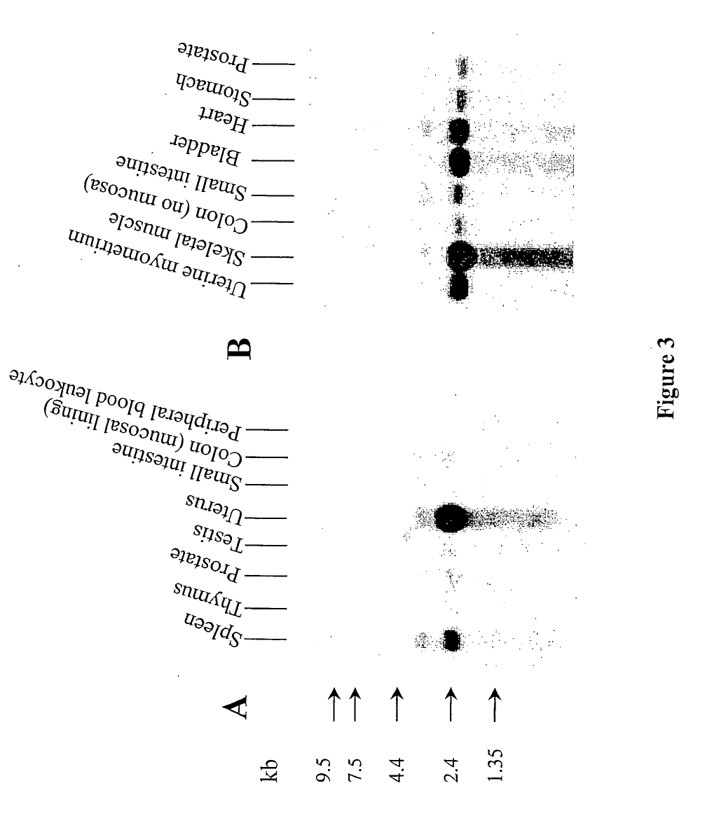

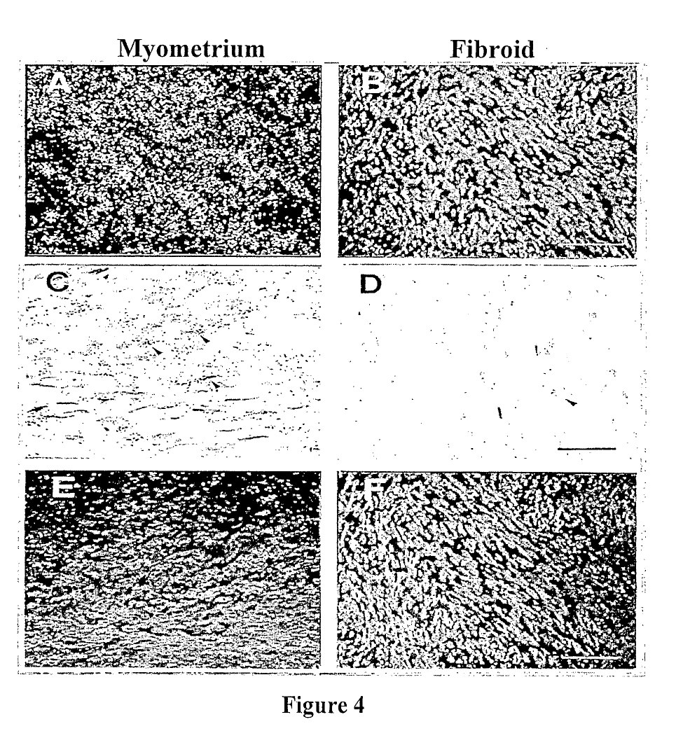

A relatively high level of Cyr61 expression was observed in spleen when compared to the uterus (FIG. 3A). Furthermore, in addition to the uterine myometrium, analysis of other human muscle tissues revealed high basal expression in skeletal muscle, heart and bladder while relatively lower levels were detected in colon, small intestine, stomach, and prostate (FIG. 3B). Therefore, high constitutive expression of Cyr61 appears to be a characteristic feature of organs such as the heart, bladder, and uterus that are comprised primarily of smooth and skeletal muscle cells. In order to determine the precise cell types in which Cyr61 is expressed in the uterus, additional in situ hybridization experiments were performed. In 6 out of 6 patients high levels of Cyr61 mRNA were detected in myometrial cells (FIGS. 4A and C). However, Cyr61 transcripts were dramatically decreased in leiomyoma smooth muscle cells (FIGS. 4B and D) from the same 6 patients. The signal from control slides hybridized with the sense probe gave no apparent signals (FIG. 4E and F). High basal levels of Cyr61 transcripts were also observed in stromal but not vascular endothelial or glandular epithelial cells in the uterus (data not shown). High basal expression of Cyr61 is primarily confined to uterine smooth muscle cells in healthy myometrium while in leiomyomas it is absent.

Tissue treatment with sex steroids and growth factors Tissue specimens obtained as described above were immediately minced into 1-2 mm pieces using sterilized scalpels and forceps and placed in phenol-red free DMEM/Ham's F12 containing antifingal and antibiotic agents only. Samples were treated ex vivo with either 10 nM E₂, 10 nM R5020, a combination of 10 μM E₂ and 10 nM R5020, 1 μM ICI 182,780 (ICI), a combination of 10 nM E, and 1 μM ICI 10 ng/ml bFGF, 10% charcoal stripped serum (CSS), or ethanol vehicle for 1 h at 37° C. under 95% air/5% CO₂. Treated tissue specimens were harvested and snap frozen in liquid nitrogen prior to RNA isolation and Northern blotting.

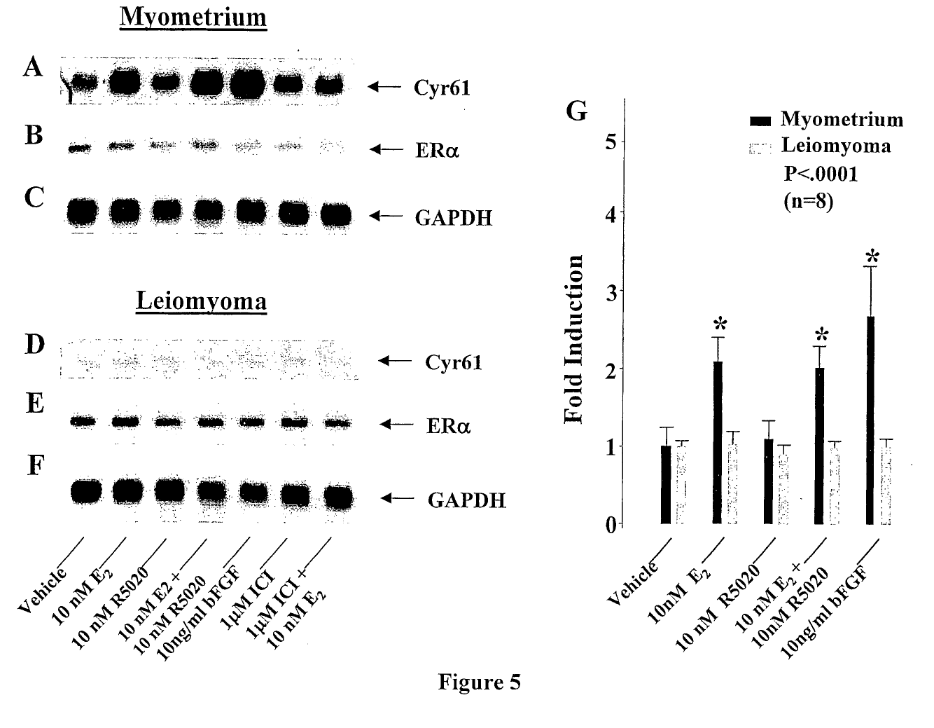

Freshly obtained leiomyoma and matched myometrial explants (n=8) were treated ex vivo with 10 nM E₂, 10 nM R5020, or a combination of 10 nM E₂ and 10 nM R5020. As a positive control, explants were stimulated with 10 ng/ml bFGF which induces Cyr61 in cell types such as murine and human fibroblasts (Nathans et al. Cold Spring Harbor Symp. Quant. Biol., 1988, 53:893-900). E₂ treatment resulted in a greater than 2 fold increase in Cyr61 transcript levels within 1 h in myometrial tissue, whereas the synthetic progesterone receptor agonist, R5020, had no effect on Cyr61 expression, nor did it synergize with E₂ (FIGS. 5A and G). The E₂ mediated induction of Cyr61 was ER dependent as it was completely inhibited by the pure ER antagonist, ICI 182,780 (FIG. 5A). Furthermore, Cyr61 expression was enhanced greater than 3 fold when myometrial explants were treated with either bFGF (FIGS. 5A and G) or serum (data not shown) for 1 h. However, neither E₂ nor bFGF was able to upregulate Cyr61 in leiomyoma tissues as observed in myometrial controls (FIGS. 5D and G). The latter phenomenon was not due to the lack of ERα expression which was consistently 2 fold higher in leiomyoma explants when compared to autologous myometrium (FIGS. 5B and E). Therefore, in addition to bFGF and serum, Cyr61 is rapidly induced by 17β-estradiol in human myometrial tissue but not in leiomyoma tumors.

Protocol for Synthesis and Purification of Recombinant Human Cyr61. The SmaI-HindIII fragement (nucleotides 100-1649) of the human Cyr61 cDNA, which encompasses the entire open reading frame, was cloned into pBlueBac4 bacluovirus expression vector (Invitrogen). Recombinant baculovirus clones were obtained, plaque-purified and amplified through three passages of Sf9 insect cell infection as described (Summers and Smith, Tex. Agric. Exp. Stn. Bull.: 1555: 1-55 (1997)). Sf cells were seeded at 2-3×10⁶ cells/P150 in serum-free sf900-II medium as monolayer cultures and were seeded at 2-3×10⁶ cells/P150 in serum-free sf900-II medium as monolayer cultures and were infected with 5 plaque forming units (PFU) of recombinant virus per cell. The conditioned medium (comprising recombinant human Cyr61 protein) was collected 48 and 96 h post-infection, cleared by centrifugation (15,000×g for 5 minutes) and adjusted to 50 mM morpholineethansulfonic acid (MES), pH=6.01 mM phenylmethylsulfonyl fluoride (PMSF) and 1 nM EDTA, pH=8. The medium was mixed with Sepharose S beads equilibrated with loading buffer (50 mM MES, pH 6.01 mM PMSF, 150 mM NaCl) at 5 ml of Sepharose S/5001 ml of conditioned medium and the proteins were allowed to bind to the Sepharose S in a batch at 4° C. overnight with gentle stirring. Sepharose S beads were collected by sedimentation without stirring for 20 min. and applied to a column. The column was washed with six volumes of the loading buffer adjusted to 0.3M NaCl and the bound proteins were eluted from the column with a step gradient of NaCl (0.4-0.8M) in the loading buffer.

Statistical analysis Values derived from densitometric measurements of RNA bands detected on Northern blots were analyzed using SAS statistical software (SAS Inc., Cary, N.C.) for significance using an one-way analysis of variance (ANOVA) for a factorial experimental design. The multiconiparison significance level for the one-factor analysis of variance was 0.05. If significance was achieved by one-way analysis, post-ANOVA comparison of means was performed using Scheffe' F. tests (Norman and Streiner, Biostatistics The Bare Essentials. St. Louis, Mo.: Mosby Press, 1994, 58 pp).

The patents, applications, test methods, and publications mentioned herein are hereby incorporated by reference in their entirety.

Many variations of the present invention will suggest themselves to those skilled in the art in light of the above detailed description. All such obvious variations are within the full intended scope of the appended claims.

We claim:

1. A method for inhibiting proliferation of uterine leiomyoma, said method comprising increasing the level of mRNA encoding Cyr61 in said leiomyoma tissue.

2. A method for inhibiting proliferation of uterine leiomyoma, said method comprising increasing the translation of Cyr61 mRNA in said leiomyoma tissue.

3. A method for inhibiting proliferation of uterine leiomyoma, said method comprising upregulating the expression of Cyr61 protein in said leiomyoma tissue.

4. A method for inhibiting proliferation of uterine leiomyoma, said method comprising increasing the activity of Cyr61 protein in said leiomyoma tissue.

5. A method for preventing uterine leiomyoma in normal myometrial tissue, said method comprising maintaining a uterine leiomyoma preventing level of mRNA encoding Cyr61 in said myometrial tissue.

6. A method for preventing uterine leiomyoma formation in normal myometrial tissue, said method comprising maintaining a uterine leiomyoma preventing level of translation activity of Cyr61 mRNA in said myometrial tissue.

7. A method for preventing uterine leiomyoma formation in normal myometrial tissue, said method comprising maintaining a uterine leiomyoma preventing level of expression of Cyr61 protein in said myometrial tissue.

8. A method for preventing uterine leiomyoma formation in normal myometrial tissue, said method comprising maintaining a uterine leiomyoma preventing level of activity of Cyr61 protein in said myometrial tissue.

9. A method for preventing uterine leiomyoma formation in normal myometrial tissue, said method comprising maintaining a uterine leiomyoma preventing level of affinity of Cyr61 protein for basic fibroblast growth factor or heparin binding epidermal growth factor in said myometrial tissue.

10. An antibody which binds to Cyr61.

11. An antibody as defined in claim 10, which selectively recognizes amino acids 371-381 of the amino acid sequence depicted in FIG. 7.

12. An antibody as defined in claim 10, which is chimeric.

13. An antibody as defined in claim 10, which is anti-idiotypic.

14. An antibody as defined in claim 13, which is conjugated to a pharmaceutically active compound.

15. An antibody as defined in claim 14, wherein said pharmaceutically active compound comprises calicheamicin.

16. An antibody as defined in claim 10, which is a monoclonal antibody.

17. An antibody as defined in claim 16, which is humanized.

18. An antibody as defined in claim 16, which is chimeric.

19. An antibody as defined in claim 16, which is anti-idiotypic.

20. An antibody as defined in claim 19, which is conjugated to a pharmaceutically active compound.

21. An antibody as defined in claim 20, wherein said pharmaceutically active compound comprises calicheamicin.

22. A method for diagnosing uterine leiomyomas, said method comprising comparing the level of Cyr61 present in suspect myometrium tissue to the level of Cyr61 in normal myometrium tissue autologous to said suspect myometrium tissue, whereby a lower level of Cyr61 in said suspect tissue than the level of Cyr61 in said normal tissue indicates that said suspect tissue comprises uterine leiomyoma.

23. The method as defined in claim 22, wherein said level of Cyr61 is determined by exposing said suspect tissue and said normal tissue to a Cyr61 antibody that selectively recognizes the Cyr61 protein and comparing the amount of antibody bound by each tissue, whereby a lower level of antibody bound by said suspect tissue than the level of antibody bound by said normal tissue indicates that said suspect tissue comprises uterine leiomyoma.

24. A method for screening for a compound which inhibits proliferation or prevents formation of uterine leiomyoma, said method comprising comparing a first amount of Cyr61 expressed by leiomyoma cells exposed to said compound to a second amount of Cyr61 expressed by said uterine leiomyoma cells that have not been exposed to said compound; whereby a greater first amount than said second amount indicates that said compound may inhibit or prevent uterine leiomyoma.

25. A transgenic non-human animal having a uterus, said animal comprising DNA which can be induced to overexpress Cyr61 in said uterus.

26. A transgenic non-human animal as defined in claim 25, wherein the DNA is human.

27. A transgenic non-human animal as defined in claim 27, wherein the animal is mouse.

28. A kit for diagnosing uterine leiomyoma, said kit comprising an antibody as defined in claim 10.

29. A method for screening compounds that regulate Cyr61 mRNA transcription through a receptor, said method comprising comparing the level of Cyr61 mRNA in a first population of cells, sufficient to transcribe a detectable amount of mRNA encoding Cyr61, when said cells are contacted with a test compound to the level of Cyr61 mRNA in a second population of cells, sufficient to transcribe a detectable amount of mRNA encoding Cyr61, not contacted with said test compound, whereby a higher level of mRNA encoding Cyr61 in said first population of cells than the level of mRNA encoding Cyr61 in said second population of cells indicates that said test compound may regulate Cyr61 mRNA transcription.

30. A method for detecting the presence of uterine leiomyoma, said method comprising comparing the level of Cyr61 mRNA isolated from suspect uterine leiomyoma tissue to the level of Cyr61 mRNA isolated from normal myometrium tissue; wherein a lower level of Cyr61 mRNA from said suspect uterine leiomyoma tissue than the level of Cyr61 mRNA from said normal tissue indicates the presence of uterine leiomyoma.

31. A method for detecting the presence of uterine leiomyoma, said method comprising comparing the level of Cyr61 in suspect uterine leiomyoma tissue to the level of Cyr61 protein in normal myometrium tissue; wherein a lower level of Cyr61 protein in said suspect tissue than the level of Cyr61 protein in said normal tissue indicates the presence of uterine leiomyoma.

32. An antibody as defined in claim 10, which is conjugated to an anti-leiomyoma agent.

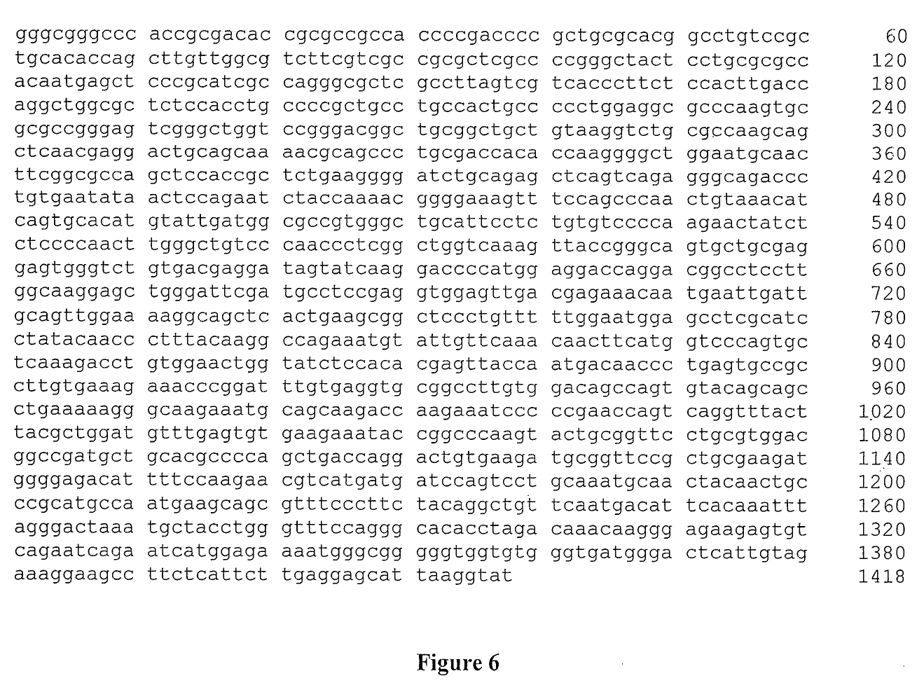

33. An expression vector comprising the nucleic acid as depicted in FIG. 6 operably associated with an expression control sequence.

34. An expression vector as defined in claim 33, wherein said expression control sequence is an estrogen response element.

35. An expression vector as defined in claim 33, wherein said expression control sequence is a basic fibroblast growth factor response element.

36. A pharmaceutical composition comprising an expression vector as defined in claim 33 in an amount effective to express a therapeutically effective amount of Cyr61.

37. A method for preventing uterine leiomyoma formation in normal myometrial tissue, said method comprising administering a pharmaceutical composition as defined in claim 36 to a subject in whom prevention of uterine leiomyoma is desired.

38. A method for inhibiting proliferation of uterine leiomyoma, said method comprising administering a pharmaceutical composition as defined in claim 36 to a subject in whom inhibiting the proliferation of uterine leiomyoma is desired.

39. A pharmaceutical composition comprising Cyr61 protein or a fragment thereof as depicted by the amino acid sequence in FIG. 7.

40. A method for preventing uterine leiomyoma formation, said method comprising administering the pharmaceutical composition as defined in claim 39 to a subject in whom prevention of uterine leiomyoma is desired.

41. A method for inhibiting proliferation of uterine leiomyoma, said method comprising administering a pharmaceutical composition as defined in claim 39 to a subject in whom inhibiting the proliferation of uterine leiomyoma is desired.

42. A method for inhibiting proliferation of uterine leiomyoma, said method comprising administering to a subject an amount of a compound effective to stimulate the synthesis of mRNA encoding Cyr61 in leiomyoma tissue.

43. A method as defined in claim 42, wherein said compound is an estrogen receptor antagonist.

44. A method for inhibiting proliferation of uterine leiomyoma, said method comprising administering to a subject, an amount of a compound effective to stimulate the translation of mRNA encoding Cyr61 in leiomyoma tissue.

45. A method as defined in claim 44, wherein said compound is an estrogen receptor antagonist.

46. A method for inhibiting proliferation of uterine leiomyoma, said method comprising administering to a subject, an amount of a compound effective to upregulate the expression of Cyr61 protein in leiomyoma tissue.

47. A method as defined in claim 46, wherein said compound is an estrogen receptor antagonist.

48. A method for inhibiting proliferation of uterine leiomyoma, said method comprising administering to a subject an amount of a compound effective to increase the activity of Cyr61 protein in leiomyoma tissue.

49. A method as defined in claim 48, wherein said compound is an estrogen receptor antagonist.

50. A method for preventing uterine leiomyoma formation in normal myometrial tissue, said method comprising administering to a subject an amount of a compound effective to maintain a uterine leiomyoma preventing level of synthesis of mRNA encoding Cyr61 in said myometrial tissue.

51. A method as defined in claim 50, wherein said compound is an estrogen receptor antagonist.

52. A method for preventing uterine leiomyoma formation in normal myometrial tissue, said method comprising administering to a subject, an amount of a compound effective to maintain a uterine leiomyoma preventing level of translation activity of Cyr61 mRNA in said myometrial tissue.

53. A method as defined in claim 52, wherein said compound is an estrogen receptor antagonist.

54. A method for preventing uterine leiomyoma formation in normal myometrial tissue, said method comprising administering to a subject, an amount of a compound effective to maintain a uterine leiomyoma preventing level of expression of Cyr61 protein in leiomyoma tissue.

55. A method as defined in claim 54, wherein said compound is an estrogen receptor antagonist.

56. A method for preventing uterine leiomyoma formation in normal myometrial tissue, said method comprising administering to a subject an amount of a compound effective to maintain a uterine leiomyoma preventing level of activity of Cyr61 protein in said myometrial tissue.

57. A method as defined in claim 56, wherein said compound is an estrogen receptor antagonist.

58. A method for preventing uterine leiomyoma formation in normal myometrial tissue, said method comprising administering to a subject an amount of a compound effective to maintain a uterine leiomyoma preventing level of affinity of Cyr61 protein for basic fibroblast growth factor or heparin binding epidermal growth factor in said myometrial tissue.

59. A method as defined in claim 58, wherein said compound is an estrogen receptor antagonist.

60. A method as defined in claim 42, wherein said compound also downregulates the synthesis of mRNA encoding at least one member selected from the group consisting of IGF I and IGF II in leiomyoma tissue.

61. A method as defined in claim 44, wherein said compound also downregulates the translation of mRNA encoding at least one member selected from the group consisting of IGF I and IGF II in leiomyoma tissue.

62. A method as defined in claim 46, wherein said compound also downregulates the expression of protein encoding at least one member selected from the group consisting of IGF I and IGF II in leiomyoma tissue.

63. A method as defined in claim 48, wherein said compound also decreases the activity of at least one member selected from the group consisting of IGF I and IGF II in leiomyoma tissue.

64. A method as defined in claim 50, wherein said compound also downregulates the synthesis of mRNA encoding at least one member selected from the group consisting of IGF I and IGF II in said myometrial tissue.

65. A method as defined in claim 52, wherein said compound also downregulates the translation of mRNA encoding at least one member selected from the group consisting of IGF I and IGF II in said myometrial tissue.

66. A method as defined in claim 54, wherein said compound also downregulates the expression of at least one member selected from the group consisting of IGF I and IGF II in said myometrial tissue.

67. A method as defined in claim 56, wherein said compound decreases the activity of at least one member selected from the group consisting of IGF I and IGF II in said myometrial tissue.

68. A method as defined in claim 42, wherein said compound also downregulates the synthesis of mRNA encoding at least one member selected from the group consisting of basic fibroblast growth factor and heparin binding epidermal growth factor in leiomyoma tissue.

69. A method as defined in claim 44, wherein said compound also downregulates the translation of mRNA encoding at least one member selected from the group consisting of basic fibroblast growth factor and heparin binding epidermal growth factor in leiomyoma tissue.

70. A method as defined in claim 46, wherein said compound also downregulates the expression of at least one member selected from the group consisting of basic fibroblast growth factor and heparin binding epidermal growth factor in leiomyoma tissue.

71. A method as defined in claim 48, wherein said compound decreases the activity of at least one member selected from the group consisting of basic fibroblast growth factor and heparin binding epidermal growth factor in leiomyoma tissue.

72. A method as defined in claim 50, wherein said compound also downregulates the synthesis of mRNA encoding at least one member selected from the group consisting of basic fibroblast growth factor and heparin binding epidermal growth factor in said myometrial tissue.

73. A method as defined in claim 52, wherein said compound also downregulates the translation of mRNA encoding at least one member selected from the group consisting of basic fibroblast growth factor and heparin binding epidermal growth factor in said myometrial tissue.

74. A method as defined in claim 54, wherein said compound also downregulates the expression of at least one member selected from the group consisting of basic fibroblast growth factor and heparin binding epidermal growth factor in said myometrial tissue.

75. A method as defined in claim 56, wherein said compound decreases the activity of at least one member selected from the group consisting of basic fibroblast growth factor and heparin binding epidermal growth factor in said myometrial tissue.

76. A method as defined in claim 60, wherein the synthesis of mRNA is downregulated by antisense nucleic acid.

77. A method as defined in claim 64, wherein the synthesis of mRNA is downregulated by antisense nucleic acid.

78. A method as defined in claim 68, wherein the synthesis of mRNA is downregulated by antisense nucleic acid.

79. A method as defined in claim 72, wherein the synthesis of mRNA is downregulated by antisense nucleic acid.

80. A method for inhibiting proliferation of uterine leiomyoma, said method comprising administering to a subject an amount of a compound effective to modulate Cyr61 protein binding to an integrin receptor.

81. A method for preventing uterine leiomyoma formation, said method comprising administering to a subject an amount of a compound effective to modulte Cyr61 protein binding to an integrin receptor.

82. A method for inhibiting proliferation of uterine leiomyoma, said method comprising increasing the level of Cyr61 in leiomyoma tissue.

183. A method for preventing uterine leiomyoma formation, said method comprising increasing the level of Cyr61 in leiomyoma tissue.

184. A pharmaceutical composition as defined in claim 39, wherein said fragment retains Cyr61 functional activity.
